(19) United States
(12) Patent Application Publication
(10) Pub. No.: US 2007/0148664 A1
(43) Pub. Date: Jun. 28, 2007

(54) Modulation of skin color

(75) Inventors: Krishna Pant, San Jose, CA (US); Renee Stokowski, San Jose, CA (US); David R. Cox, Belmont, CA (US); Martin Richard Green, Cambridge (GB); Franciscus Gerrit Van Der Ouderaa, Sharonbrook (GB); Rebecca Susan Ginger, Mears Ashby (GB); Amelia Clare Fereday, Stanwick (GB); Wendy Filsell, Wellingborough (GB); Carl Dudley Jarman, Wellingborough (GB); Anthony Dadd, Sharnbrook (GB)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: Perlegen Sciences, Inc., Mountain View, CA (US); Unilever PLC

(21) Appl. No.: 11/515,497

(22) Filed: Sep. 1, 2006

Related U.S. Application Data
(60) Provisional application No. 60/713,879, filed on Sep. 2, 2005.

(51) Int. Cl. C12Q 1/68; G01N 33/53

(52) U.S. Cl. 435/6; 435/7.1

(57) ABSTRACT

The invention provides a collection of polymorphic sites associated with variations in human skin color, and genes containing or proximal to the sites.

### CROSS-REFERENCE TO RELATED APPLICATION

The present application is a nonprovisional and claims the benefit under 35 USC 119(e) of 60/713,879, Sep. 2, 2005, which is incorporated by reference in its entirety for all purposes.

### BACKGROUND OF THE INVENTION

The skin is the body's largest organ and has roles in themoregulation, protection from physical and chemical injury, protection from invasion by microorganisms, and manufacture of vitamin D. There is a wide continuous range of human skin color, which can be correlated with climates, continents, and cultures. For example, skin color is darkest in those living at the equator and then gradually lightens with increasing latitude in both the northern and southern hemispheres. The darker skin color at the equator is thought to provide protection from heat and ultraviolet irradiation. The lighter skin color away from the equator is thought to provide protection from frost bite and to facilitate synthesis of vitamin D. The primary basis for skin color appears to be genetic in that dark-skinned persons transplanted to higher latitudes show little lightening of skin color and light-skinned Europeans transplanted to equatorial latitudes show little darkening. Light-skinned individuals transplanted to equatorial regions are particularly susceptible to uv-induced diseases such as skin cancer, folic acid deficiency and suppressed immune systems.

The principal pigments responsible for skin color are carotene, hemoglobin and in particular melanin. Melanin is the primary determinant of variability. Melanin has a dark brown/purple/black color. The amount, density and distribution of melanin controls variability of human skin color. Carotene is sometimes associated with pathological or abnormal skin coloration. Hemoglobin is the primary protein constituent of red blood cells. Oxygenated hemoglobin has a reddish hue and produces a pinking tint to a lightly pigmented skin. Deoxygenated hemoglobin has a purple color and produces a bluish tint to light pigmented skin when deprived of oxygen.

Melanin is synthesized by melanocytes, and injected into surrounding keratinocytes. The metabolic pathway to melanin is complex starting with oxidation of the amino acid tyrosine by the copper containing enzyme tyrosinase to dihydroxphenylallanine and then to dopaquinone. Mutations in the tyrosinase enzyme is known to result in one form of albinism. Dopaquinone undergoes a series of non-enzymatic reactions and rearrangements forming molecules that are co-polymerized to form either eumelanin, the dark brown-purple-black compound found in skin or hair, or phaeomelanin, a yellow-red pigment present in red hair.

Sunlight can temporarily change skin color (i.e., tanning) by a two-stage process. Immediate tanning is a transient browning tan occurring within 1-2 hr of exposure. Such tanning is due to photooxidation of melanin or other epidermal elements. Delayed tanning is a more prolonged browning occurring 2-3 days after exposure. This tanning is due to enhanced synthesis of melanin and consequently deposits of melanin.

The genetic basis for the color variation is poorly understood. Much of the available information comes from studies of variations in mouse coat color. It is not known how many genes are involved in determining skin pigmentation in humans or what genetic variations with in them are responsible for different phenotypes.

### SUMMARY OF THE CLAIMED INVENTION

The invention provides a method of screening a compound for activity in modulating tissue color. The method comprises determining whether a compound binds to, modulates expression of, or modulates the activity of a polypeptide encoded by a gene shown in Table 2, column 3, 5 or 7.

The invention further provides a method of modulating tissue color of a subject. The method comprises administering to the subject an effective amount of a compound that modulates tissue color of the subject.

The invention further provides a transgenic nonhuman animal having a genome comprising an exogenous gene shown in Table 2, column 3, 5 or 7, wherein the gene is expressed and modulates skin color of the nonhuman animal relative to a control nontransgenic animal.

The invention further provides a transgenic nonhuman animal having a genome in which a nonhuman homolog of a human gene shown in Table 2, column 3, 5 or 7 is disrupted, whereby the disrupted gene modulates skin color of the transgenic nonhuman animal relative to a control nontransgenic animal.

The invention further provides a method of polymorphic profiling an individual. Such a method comprises determining a polymorphic profile in at least two but no more than 1000 different haplotype blocks, and at least two of the haplotype blocks each overlapping at least one gene shown in Table 2, columns 3, 5 or 7.

The invention further provides a method of selecting a treatment to modulate tissue color of an individual, comprising determining a polymorphic profile in at least one haplotype block, overlapping at least one gene shown in Table 2, columns 3, 5 or 7; and selecting a treatment to modulate tissue color of the individual based on the polymorphic profile.

The invention further provides for the use of a gene shown in Table 2, columns 3, 5 or 7, a protein encoded by the gene, or of a polymorphic site in the gene or in linkage disequilibrium therewith in the modulation of skin color.

The invention further provides an isolated protein encoded by an SLC24A5 gene in which codon 111 (measured from the mature N-terminus) is occupied by threonine or alanine.

The invention further provides an isolated protein encoded by an ATP8B4 gene.

The invention further provides a method of screening a compound for activity in treating cancer. The method comprise determining whether a compound binds to, modulates expression of, or modulates the activity of a polypeptide encoded by a gene shown in Table 2, column 3, 5 or 7.

The invention further provides a method of effecting prophylaxis or treatment of cancer in a subject having or at risk of cancer. The method comprises administering to the subject an effective amount of an agent. The agent is preferably selected from the group consisting of an antibody that specifically binds to a protein encoded by a gene shown in Table 2, column 3, 5 or 7; a zinc finger protein that modulates expression of a gene shown in Table 2, column 3, 5 or 7; an siRNA or antisense RNA, RNA complementary to a regulatory site, or ribozyme that inhibits expression of a gene shown in Table 2, column 3, 5 or 7; whereby the agent effects prophylaxis or treatment of cancer in the subject.

The invention further provides a transgenic nonhuman animal having a genome comprising an exogenous gene shown in Table 2, columns 3, 5 or 7, wherein the gene is expressed and disposes the nonhuman animal to cancer relative to a control nontransgenic animal.

The invention further provides a transgenic nonhuman animal having a genome in which a nonhuman homolog of a human gene shown in Table 2, columns 3, 5 or 7 is disrupted, whereby the disrupted gene disposes the transgenic nonhuman animal to cancer relative to a control nontransgenic animal.

The invention further provides a method of determining susceptibility to cancer. The method comprises determining a polymorphic profile in at least one haplotype block overlapping at least one gene selected shown in Table 2, columns 3, 5 or 7; a difference in polymorphic profile relative to an undiseased individual indicating susceptibility to cancer.

The invention further provides for the use of a gene shown in Table 2, column 3, 5 or 7, or a protein encoded by the gene or a SNP in the gene or in linkage disequilibrium therewith for in the prognosis, diagnosis, prophylaxis or treatment of cancer.

The invention further provides a method of screening a compound for activity in treating hypertension. The method comprises determining whether a compound binds to, modulates expression of, or modulates the activity of a polypeptide encoded by a gene shown in Table 2, column 3, 5 or 7.

The invention further provides a method of effecting prophylaxis or treatment of hypertension in a subject having or at risk of hypertension. The method comprises administering to the subject an effective amount of a compound. The compound is preferably selected from the group consisting of: an antibody that specifically binds to a protein encoded by a gene shown in Table 2, column 3, 5 or 7; a zinc finger protein that modulates expression of a gene shown in Table 2, column 3, 5 or 7; an siRNA, antisense RNA, RNA complementary to a regulatory site, or ribozyme that inhibits expression of a gene a gene shown in Table 2, column 3, 5 or 7. The agent effects prophylaxis or treatment of hypertension in the subject.

The invention further provides a transgenic nonhuman animal having a genome comprising an exogenous gene shown in Table 2, column 3, 5 or 7; wherein the gene is expressed and disposes the nonhuman animal relative to hypertension relative to a control nontransgenic animal.

The invention further provides a transgenic nonhuman animal having a genome in which a nonhuman homolog of a human gene shown in Table 2, column 3, 5 or 7 is disrupted, whereby the disrupted gene disposes the transgenic nonhuman animal to hypertension relative to a control transgenic animal.

The invention further provides a method of determining susceptibility to hypertension. The method comprises determining a polymorphic profile in at least one haplotype block overlapping at least one gene shown in Table 2, column 3, 5 or 7; a difference in polymorphic profile relative to an undiseased individual indicating susceptibility to hypertension.

The invention further provides for use of a gene shown in Table 2, columns 3, 5 or 7 or a protein encoded thereby, or a polymorphism within the gene or in linkage disequilibrium therewith in the prognosis, diagnosis, prophylaxis or treatment of hypertension.

The invention further provides a method of expression profiling. The method comprises determining expression levels of at least 2 and no more than 10,000 genes in a subject, wherein at least two of the genes are from Table 3, the expression levels forming an expression profile.

### DEFINITIONS

A polymorphic site is a locus of genetic variation in a genome. A polymorphic site is occupied by two or more polymorphic forms (also known as variant forms or alleles). A single nucleotide polymorphic site (SNP) is a variation at a single nucleotide.

The term “haplotype block” refers to a region of a chromosome that contains one or more polymorphic sites (e.g., 1-10) that tend to be inherited together (i.e., are in linkage disequilibrium) (see Patil, et al., Science, 294:1719-1723 (2001); US 20030186244)). In other words, combinations of polymorphic forms at the polymorphic sites within a block cosegregate in a population more frequently than combinations of polymorphic sites that occur in different haplotype blocks. In some embodiments, haplotype blocks do not overlap one another. In some embodiments, a haplotype block is also a linkage disequilibrium bin (LD bin).

The term “haplotype pattern” refers to a combination of polymorphic forms that occupy polymorphic sites, usually SNPs, on a single DNA strand. In some embodiments, a haplotype pattern contains only alleles of SNPs that are in a single haplotype block. For example, the combination of variant forms that occupy all the polymorphisms within a particular haplotype block on a single strand of nucleic acid is collectively referred to as a haplotype pattern of that particular haplotype block. Many haplotype blocks are characterized by four or fewer haplotype patterns in at least 80% of individuals (e.g., which can be measured using a representative sample of individuals from the world). The identity of a haplotype pattern can often be determined from one or more haplotype determining polymorphic sites without analyzing all polymorphic sites constituting the pattern.

The term “linkage disequilibrium” refers to the preferential segregation of a particular genetic locus with another genetic locus more frequently than expected by chance. For example, linkage disequilibrium can refer to the preferential segregation of a particular polymorphic site with another polymorphic site at a different chromosomal location, or the preferential segregation of a particular genetic locus (e.g., polymorphism) with a gene. Linkage disequilibrium can also refer to a situation in which a phenotypic trait displays preferential segregation with a particular polymorphic form or another phenotypic trait more frequently than expected by chance.

A polymorphic site is proximal to a gene if it occurs within the intergenic region between the transcribed region of the gene and that of an adjacent gene. Usually, proximal implies that the polymorphic site occurs closer to the transcribed region of the particular gene that that of an adjacent gene. Typically, proximal implies that a polymorphic site is within 2.4 Mb and preferably within 50 kb, or 10 kb of the transcribed region. Polymorphic sites not occurring in proximal regions as defined above are said to occur in regions that are distal to the gene.

The term “specific binding” refers to the ability of a first molecule (e.g., an antibody) to bind or duplex to a second molecule (e.g., a polypeptide) in a manner such that the second molecule can be identified or distinguished from other components of a mixture (e.g., cellular extracts, total cellular polypeptides, etc.). Specific binding between two entities means a mutual affinity of at least 10⁶ M⁻¹, and usually at least 10⁷ or 10⁸ M⁻¹. The two entities also usually have at least 10-fold greater affinity for each other than the affinity of either entity for an irrelevant control.

A nonhuman homolog of a human gene is the gene in a nonhuman species, such as a mouse, that shows greatest sequence identity at the nucleic acid and encoded protein level, and higher order structure and function of the protein product similar to that of the human gene or encoded product.

The term “modulate” refers to a change such as in expression, lifespan, or function such as an increase, decrease, alteration, enhancement or inhibition of expression or activity of a gene product.

The terms “isolated” and “purified” refer to a material that is substantially or essentially removed from or concentrated in its natural environment. For example, an isolated nucleic acid is one that is separated from the nucleic acids that normally flank it or from other biological materials (e.g., other nucleic acids, proteins, lipids, cellular components, etc.) in a sample. In another example, a polypeptide is purified if it is substantially removed from or concentrated in its natural environment.

“Statistically significant” means significant at a p-value ≦0.05.

The term “comprising” indicates that other elements can be present besides those explicitly stated.

### DETAILED DESCRIPTION OF THE INVENTION

I. General

The invention provides a collection of polymorphic sites associated with variations in human skin color, and genes containing or proximal to the sites. The polymorphic sites were identified by two genetic association studies, the first between volunteers with proven ancestry from the subcontinent of India (i.e., South Asians) having either lighter or darker skin color and the second study between European Americans and African Americans with predicted relatively light and dark skin colors respectively. Most of the polymorphic sites showed similar associations in both studies.

The collection of polymorphic sites and genes has a variety of uses. The genes and encoded proteins can be used to identify compounds that modulate the expression or activity of encoded proteins. Such compounds are useful for modulating skin color. Modulating skin color is desirable both for cosmetic purposes, and for treatment of several diseases and conditions associated with skin color. The collection of genes are also useful for generating transgenic animal models of modulated skin color. These models are useful for screening drugs. The polymorphic sites are also useful in profiling individuals for susceptibility to disease, response to therapies, or amenability to treatment.

II. Polymorphic Sites and Genes

The invention provides a collection of 153 polymorphic sites (all SNPs) in the human genome, each of which has one polymorphic form associated with lighter skin and one polymorphic form associated with darker skin. The polymorphic sites are listed in Table 1. The first column of the table indicates the chromosome on which the polymorphic site is found. Many of the polymorphic sites are found on chromosome 15. The second column provides the location of the SNP (National Center of Biotechnology Information (NCBI) Build 35 of the human genome map). The third and fourth columns provide NCBI dbSNP identification numbers for each SNP. If a SNP has an RS_ID but not an SS_ID, this means that Perlegen Sciences has not submitted this SNP to dbSNP, but an existing SNP in dbSNP maps (in the Perlegen alignment process) to the same location as the Perlegen SNP. The fifth and sixth columns indicate the nucleotide base occupying the SNP with greater frequency in darker and lighter skinned volunteers, respectively. The seventh column shows a 29mer nucleic acid centered around the SNP. The 15th central position shows the two bases that can occupy the SNP in IUB-IUPAC ambiguity code. The invention also includes polymorphic sites and polymorphic forms occupying them in linkage disequilibrium with the exemplified SNPs.

Table 2 provides the genes containing the polymorphic sites shown in Table 1 or genes proximate to them. Some polymorphic sites do not occur within the transcript of a gene and thus only flanking genes (within the maximum distance of 4,000 kilobases upstream or downstream from the polymorphic site) are shown. The genes containing polymorphic sites, flanking sequences and surrounding genes in linkage disequilibrium therewith likely contain additional polymorphic sites, which are in linkage disequilibrium with the identified polymorphic sites and can be similarly used. The first and second columns of Table 2 provide the chromosome and polymorphic position as in Table 1. The third column provides the name of the gene containing the polymorphic site. Not all polymorphic sites occur within a gene. The gene names are those defined by the authorities in the field such as HUGO, or conventionally used in the art to describe the genes. GeneID numbers for these the genes at NCBI Gene database are provided in Table 3. Table 3 lists alternative names for some genes separated by a “/”. The gene TAZ also known as WWTR1 is the gene present on chromosome 3. Only one name is used for genes in other tables. The fourth column of Table 2 provides the location in the gene transcript of a polymorphic site (e.g., intron, exon). The term “non-synonymous” means the variation between the two polymorphic forms occupying a polymorphic site has a corresponding change at the amino acid level in the protein encoded by the gene. The term “synonymous” means the variation between the two polymorphic forms occupying a polymorphic site does not have a corresponding change at the amino acid level in the protein encoded by the gene. Columns 5-8 provide the identity of genes on either side of (but not containing) a polymorphic site, and the distance from the gene. The distance is measured in kb between the ends of the respective transcript encoding regions.

The analysis identified 29 discrete genes containing polymorphisms associated with skin color. MATP and TYR were already known to associated. The analysis identified a collection of additional genes flanking (in distance equal or less than 4,000 kb upstream or downstream) polymorphic sites of the invention without containing them.

Several genes containing polymorphic sites showing particularly strong associations with skin color are described below. SLC24A5 is a solute carrier family 24 (sodium/potassium/calcium exchanger), member 5, located on chromosome 15. This family of K⁺-dependent Na⁺/Ca²⁺ exchangers catalyze the electrogenic counter transport of Na⁺ for Ca²⁺ and K⁺, so this gene is involved in Ca²⁺ uptake/efflux of cells. The tissue expression by this gene is not well characterized, but the mRNA is found in skin cDNA libraries. One SNP at position 46213776 falls within the coding sequence of the gene and causes a nonsynonymous amino acid change of alanine to threonine at codon 111. This SNP was only genotyped in the replication populations (see Examples), but gives an allele frequency difference (delta-p) of 39% between lighter and darker skinned volunteers. The affected amino acid falls within a conserved domain of the Na⁺/Ca²⁺ exchanger family, but is not a conserved amino acid. Another SNP at position 46179457 with a delta-p of 43% between lighter and darker skinned volunteers, and a delta-p of 74% between European and African Americans, is located 21 kb from this gene.

The effect of SLC24A5 in skin color can be rationalized by its role in mediating calcium uptake/efflux in human melanocytes, and thus regulating melanin production. Transport of extracellular L-phenylalanine, its intracellular metabolism to L-tyrosine via intracellular phenylalanine hydroxylase, and incorporation into melanin have been reported to be coupled to calcium uptake/efflux in melanocytes (Biochem Biophys Res Commun. 1999 Aug. 27; 262(2):423-8). Calcium has been reported to be a key regulator of melanocyte function (Buffy et al., Pigment Cell Research 6, 385-393 (1993)). Others have reported melanin granules and melanosomes regulate calcium concentrations in the melanocytes of retinal pigment epithelium (Cell Calcium. 2000 April 27(4):223-9; Pigment Cell Res. 1990 Sep.; 3(3):141-5). Melanocytes are also found in the hair follicle, inner ear and in the iris of the eye.

Another transporter found by the present analysis to be associated with skin color include SLC12A1, solute carrier family 1. The sodium-potassium-chloride co-transporter isoform 2 is kidney-specific and is found on the apical membrane of the thick ascending limb of Henley's loop and the macula dense. It accounts for most of the NaCl resumption with the stoichiometry for Na:K:Cl of 1:1:2: and is sensitive to such diuretics as furosemide and bumetanide. SLC12A1 may indirectly affect melanocyte function through influence on plasma potassium levels.

Another transporter associated with skin color is a sodium/potassium/chloride transporter, SLC7A2, solute carrier family 7 (cationic amino acid transporter, y+ system), member 2. This transporter is expressed in keratinocytes. A role of the transporter in skin color can be rationalized as controlling L-arginine uptake. L-arginine is essential for inducible nitric oxide synthase and arginase enzymes, which modulate proliferation and differentiation of epidermal cells.

Another transporter associated with skin color is SLC27A2, solute carrier family 27 (fatty acid transporter), member 2 and ABCC9, an ATP-binding cassette, sub-family C (CFTR/MRP), member 9. Long chain fatty acids (LCFAs) are an important source of energy for most organisms. They also function as blood hormones, regulating key metabolic functions such as hepatic glucose production. Another gene with related function associated with skin color is ACSL4. This gene encodes fatty acid-CoA ligase 4. A mutation in this gene has previously been associated with nonspecific X-linked mental retardation.

Another transporter associated with skin color is ATP8B4. This transporter is believed to be a phospholipid-transporting ATPase and a lipid flipase.

dUTP pyrophosphatase, also known as dUTPase, is also located on chromosome 15. This gene catalyses the reaction: dUTP+H2O=dUMP+diphosphate. This gene is present in skin cDNA libraries and seems to be ubiquitously expressed (Proc. Natl. Acad. Sci. USA. 1992 Sep. 1; 89(17):8020-4). A SNP at position 46420445 with a delta-p of 28% between lighter and darker skinned volunteers and a delta-p of 58% between European and African Americans is found in an intron of this gene. The effect of this gene on skin color can be rationalized from reports of in vitro binding assays indicating that rat dUTPase interacts with all three murine peroxisome proliferator-activated receptors isoforms (PPARs) and blocks the formation of PPAR-RXR heterodimers, causing repression of PPAR-mediated transcriptional activation (Br J Dermatol. 2004 March; 150(3):462-8). PPARs have been reported to be expressed in human melanocytes, with activation of the PPARs inhibiting proliferation of melanocytes and stimulating melanin synthesis (J. Cell. Physiol. 2000 June; 183(3):364-72).

SHC4 (previously RALP (rai-like protein)) is also located on chromosome 15. The exact function of this gene is not known, but because it contains an src homology 2 domain and an SHC phosphotyrosine-binding domain, it is likely to be involved in a signal transduction pathway. The tissue expression of this gene is not well characterized, but the mRNA is found in skin cDNA libraries. Eleven of the SNPs in Table 2 covering 8 LD bins, fall within the introns of this gene. The most significant SNP has a delta-p of 20% between lighter and darker skinned volunteers and between European and African Americans. The other SNPs in this range have delta-p-values ranging from 10-18% between lighter and darker skinned volunteers.

GRM5 (glutamate receptor, metabotropic 5), also known as mGlu5, is located on chromosome 11. The GRM5 protein is a G protein-coupled receptor that binds L-glutamate and is part of a group that activate phospholipase C. This gene is expressed in human melanocytes (J Cell Physiol. 2000 June; 183(3):364-72). Ten SNPs from Table 2 are located in the introns of this gene, which fall into two LD bins. The most significant SNP has a delta-p of 14% between lighter and darker skinned volunteers, and a delta-p of 48% between European and African American. The other SNPs have delta-p-values ranging between 12-14% between lighter and darker skinned volunteers. The mGlu5 receptor is expressed in human melanocytes. (J. Cell. Physiol. 2000 June;183(3):364-72). The major activator of this receptor, L-glutamate, has been reported to stimulate tyrosinase activity and promote melanin synthesis in Sepia ink glands through the NMDA receptor pathway (J. Biol. Chem. 2000 Jun. 2; 275(22):16885-90). The mGlu5 and NMDA receptors are known to functionally interact in multiple cell types (Br. J. Pharmacol. 2004 July; 142(6):991-1001, Epub 2004 Jun. 21; Psychopharmacology (Berl). 2005 Feb. 22; [Epub ahead of print]: Neuropsychopharmacology. 2004 July; 29(7):1259-69).

III. Skin Color Types, Measurement of Skin Color

For purposes of screening drugs or monitoring the effect of treatments, skin color can be assessed either by observation or quantitative criteria. Human skin responses to sunlight have been classified by Fitzpatrick and can be subjectively classified into six skin types: (1) light skinned, bums easily, never tans; (2) light skinned, bums easily, tans some; (3) light skinned, bums occasionally, tans well; (4) light skinned, tans well, rarely bums, (5) brown skinned (Asian, Indo-Asian, Chinese, Japanese), tans well, bums rarely, can sunburn after prolonged exposure to UVR; (6) black skinned (Afro-Caribbean), deeply pigmented, can bum after prolonged exposure to UVR. In the U.S. roughly 25% of people are types I & II.

More recently, quantitative methods based on reflectance spectrophotometry have been applied, which allow reddening caused by inflammation and increased hemoglobin to be distinguished from darkening caused by increased melanin (Alaluf et al., Pigment Cell Res 15: 119-126 (2002); Shriver and Parra, Am. J. Phys. Anthropol. 112: 17-27 (2000); Wagner et al., Pigment Cell. Res. 15: 379-384 (2002). Individuals assesses by a quantitative method have a gradations of different skin colors. Thus, light and dark skin color are relative terms used synonymously with lighter and darker skin color to indicate individuals toward the lighter end (e.g., lightest quintile) and darker end (e.g., darkest quintile) of a range of skin color in a population.

IV. Compounds to Modulate Skin Color

A variety of compounds can be screened for capacity to modulate expression or activity of genes associated with skin color. Compounds can be obtained from natural sources, such as, e.g., marine microorganisms, algae, plants, and fungi. Alternatively, compounds can be from combinatorial libraries of agents, including peptides or small molecules, or from existing repertories of chemical compounds synthesized in industry, e.g., by the chemical, pharmaceutical, environmental, agricultural, marine, cosmeceutical, drug, and biotechnological industries. Compounds can include, e.g., pharmaceuticals, therapeutics, environmental, agricultural, or industrial agents, pollutants, cosmeceuticals, drugs, organic compounds, lipids, glucocorticoids, antibiotics, peptides, proteins, sugars, carbohydrates, and chimeric molecules.

Combinatorial libraries can be produced for many types of compounds that can be synthesized in a step-by-step fashion. Such compounds include polypeptides, proteins, nucleic acids, beta-turn mimetics, polysaccharides, phospholipids, hormones, prostaglandins, steroids, aromatic compounds, heterocyclic compounds, benzodiazepines, oligomeric N-substituted glycines and oligocarbamates. Large combinatorial libraries of compounds can be constructed by the encoded synthetic libraries (ESL) method described in Affymax, WO 95/12608; Affymax, WO 93/06121; Columbia University, WO 94/08051; Pharmacopeia, WO 95/35503; and Scripps, WO 95/30642 (each of which is incorporated herein by reference in its entirety for all purposes). Peptide libraries can also be generated by phage display methods. See, e.g., Devlin, WO 91/18980. Compounds to be screened can also be obtained from governmental or private sources, including, e.g., the National Cancer Institute's (NCI) Natural Product Repository, Bethesda, Md., the NCI Open Synthetic Compound Collection, Bethesda, Md., NCI's Developmental Therapeutics Program, or the like. For genes encoding transporters, the compounds include substrates of the transporters, and analogs of the same. For ion transporters, such as SLC24A5, compounds include diuretics. Examples of diuretics are chlorothiazide, hydrochlorothiazide, hydroflumethiazide, methyclothiazide, bendroflumethiazide, benzthiazide, cyclothiazide, polythiazide, and trichlormethiazide, chlorthalidone, indapamide, metolazone, and quinethazone. For gene ABCC9, compounds include sulfonylurea-based drugs, such as acetohexamide (Dymelor), chloropropamide (Diabinese), tolazamide (Tolinase), tolbutamide (Orinase), glimepiride (Amaryl), glipizide (Glucotrol, Glucotrol XL), glyburide (DiaBeta, Micronase, Glynase). For transporters transporting cationic amino acids and phospholipids, analogs of these natural substrates can be screened for activity as modulators of transport.

Some compounds are currently in use or for modulation of skin color such as hydroquinone, tretinoin, niacinamide and a cortisone cream. Other compounds have been approved for some indication other than modulation of skin color. Other compounds are suspected of having a role in modulation of skin color, including compounds presently in clinical trials. Some compounds are suitable for inclusion in cosmetic products.

The compounds include antibodies, both intact and binding fragments thereof, such as Fabs, Fvs, which specifically bind to a protein encoded by a gene of the invention. Usually the antibody is a monoclonal antibody although polyclonal antibodies can also be expressed recombinantly (see, e.g., U.S. Pat. No. 6,555,310). Examples of antibodies that can be expressed include mouse antibodies, chimeric antibodies, humanized antibodies, veneered antibodies and human antibodies. Chimeric antibodies are antibodies whose light and heavy chain genes have been constructed, typically by genetic engineering, from immunoglobulin gene segments belonging to different species (see, e.g., Boyce et al., Annals of Oncology 14:520-535 (2003)). For example, the variable (V) segments of the genes from a mouse monoclonal antibody may be joined to human constant (C) segments. A typical chimeric antibody is thus a hybrid protein consisting of the V or antigen-binding domain from a mouse antibody and the C or effector domain from a human antibody. Humanized antibodies have variable region framework residues substantially from a human antibody (termed an acceptor antibody) and complementarity determining regions substantially from a mouse-antibody, (referred to as the donor immunoglobulin). See Queen et al., Proc. Natl. Acad. Sci. USA 86:10029-10033 (1989) and WO 90/07861, U.S. Pat. No. 5,693,762, U.S. Pat. No. 5,693,761, U.S. Pat. No. 5,585,089, U.S. Pat. No. 5,530,101 and Winter, U.S. Pat. No. 5,225,539. The constant region(s), if present, are also substantially or entirely from a human immunoglobulin. Antibodies can be obtained by conventional hybridoma approaches, phage display (see, e.g., Dower et al., WO 91/17271 and McCafferty et al., WO 92/01047), use of transgenic mice with human immune systems (Lonberg et al., WO93/12227 (1993)), among other sources. Nucleic acids encoding immunoglobulin chains can be obtained from hybridomas or cell lines producing antibodies, or based on immunoglobulin nucleic acid or amino acid sequences in the published literature.

The compounds also include several categories of molecules known to regulate gene expression, such as zinc finger proteins, ribozymes, siRNAs and antisense RNAs. Zinc finger proteins can be engineered or selected to bind to any desired target site within a gene of the invention. An exemplary motif characterizing one class of these proteins (C₂H₂ class) is -Cys-(X)₂₋₄-Cys-(X)₁₂-His-(X)₃₋₅-His (where X is any amino acid). A single finger domain is about 30 amino acids in length, and several structural studies have demonstrated that it contains an alpha helix containing the two invariant histidine residues and two invariant cysteine residues in a beta turn co-ordinated through zinc. In some methods, the target site is within a promoter or enhancer. In other methods, the target site is within the structural gene. In some methods, the zinc finger protein is linked to a transcriptional repressor, such as the KRAB repression domain from the human KOX-1 protein (Thiesen et al., New Biologist 2, 363-374 (1990); Margolin et al., Proc. Natl. Acad. Sci. USA 91, 4509-4513 (1994); Pengue et al., Nucl. Acids Res. 22:2908-2914 (1994); Witzgall et al., Proc. Natl. Acad. Sci. USA 91, 4514-4518 (1994)). In some methods, the zinc finger protein is linked to a transcriptional activator, such as VIP16. Methods for selecting target sites suitable for targeting by zinc finger proteins, and methods for design zinc finger proteins to bind to selected target sites are described in WO 00/00388. Methods for selecting zinc finger proteins to bind to a target using phage display are described by EP.95908614.1. The target site used for design of a zinc finger protein is typically of the order of 9-19 nucleotides.

Ribozymes are RNA molecules that act as enzymes and can be engineered to cleave other RNA molecules at specific sites. The ribozyme itself is not consumed in this process, and can act catalytically to cleave multiple copies of mRNA target molecules. General rules for the design of ribozymes that cleave target RNA in trans are described in Haseloff & Gerlach, (1988) Nature 334:585-591 and Hollenbeck, (1987) Nature 328:596-603 and U.S. Pat. No. 5,496,698. Ribozymes typically include two flanking segments that show complementarity to and bind to two sites on a transcript (target subsites) of one of the genes of the invention and a catalytic region between the flanking segments. The flanking segments are typically 5-9 nucleotides long and optimally 6 to 8 nucleotides long. The catalytic region of the ribozyme is generally about 22 nucleotides in length. The MRNA target contains a consensus cleavage site between the target subsites having the general formula NUN, and preferably GUC. (Kashani-Sabet and Scanlon, (1995) Cancer Gene Therapy 2:213-223; Perriman, et al., (1992) Gene (Amst.) 113:157-163; Ruffner, et al., (1990) Biochemistry 29: 10695-10702); Birikh, et al., (1997) Eur. J. Biochem. 245:1-16; Perrealt, et al., (1991) Biochemistry 30:4020-4025). The specificity of a ribozyme can be controlled by selection of the target subsites and thus the flanking segments of the ribozyme that are complementary to such subsites. Ribozymes can be delivered either as RNA molecules or in the form of DNA encoding the ribozyme as a component of a replicable vector or in nonreplicable form as described below.

Endogenous expression of a target gene can also be reduced by delivering nucleic acids having sequences complementary to the regulatory region of the target gene (i.e., the target gene promoter and/or enhancers) to form triple helical structures which prevent transcription of the target gene in target cells in the body. See generally, Helene, (1991), Anticancer Drug Des., 6(6):569-584; Helene, et al., (1992), Ann. N.Y. Acad. Sci., 60:27-36; and Maher, (1992), Bioassays 14(12):807-815.

Antisense polynucleotides can cause suppression by binding to, and interfering with the translation of sense mRNA, interfering with transcription, interfering with processing or localization of RNA precursors, repressing transcription of niRNA or acting through some other mechanism (see, e.g., Sallenger et al. Nature 418, 252 (2002). The particular mechanism by which the antisense molecule reduces expression is not critical. Typically antisense polynucleotides comprise a single-stranded antisense sequence of at least 7 to 10 to typically 20 or more nucleotides that specifically hybridize to a sequence from mRNA of a gene of the invention. Some antisense polynucleotides are from about 10 to about 50 nucleotides in length or from about 14 to about 35 nucleotides in length. Some antisense polynucleotides are polynucleotides of less than about 100 nucleotides or less than about 200 nucleotides. In general, the antisense polynucleotide should be long enough to form a stable duplex but short enough, depending on the mode of delivery, to administer in vivo, if desired. The minimum length of a polynucleotide required for specific hybridization to a target sequence depends on several factors, such as G/C content, positioning of mismatched bases (if any), degree of uniqueness of the sequence as compared to the population of target polynucleotides, and chemical nature of the polynucleotide (e.g., methylphosphonate backbone, peptide nucleic acid, phosphorothioate), among other factors.

siRNAs are relatively short, at least partly double stranded, RNA molecules that serve to inhibit expression of a complementary mRNA transcript. Although an understanding of mechanism is not required for practice of the invention, it is believed that siRNAs act by inducing degradation of a complementary mRNA transcript. Principles for design and use of siRNAs generally are described by WO 99/32619, Elbashir, EMBO J. 20, 6877-6888 (2001) and Nykanen et al., Cell 107, 309-321 (2001); WO 01/29058. siRNAs are formed from two strands of at least partly complementary RNA, each strand preferably of 10-30, 15-25, or 17-23 or 19-21 nucleotides long. The strands can be perfectly complementary to each other throughout their length or can have single stranded 3′-overhangs at one or both ends of an otherwise double stranded molecule. Single stranded overhangs, if present, are usually of 1-6 bases with 1 or 2 bases being preferred. The antisense strand of an siRNA is selected to be substantially complementary (e.g., at least 80, 90, 95% and preferably 100%) complementary to a segment of a transcript from a gene of the invention. Any mismatched bases preferably occur at or near the ends of the strands of the siRNA. Mismatched bases at the ends can be deoxyribonucleotides. The sense strand of an siRNA shows an analogous relationship with the complement of the segment of the gene transcript of interest. siRNAs having two strands, each having 19 bases of perfect complementarity, and having two unmatched bases at the 3′ end of the sense strand and one at the 3′ end of the antisense strand are particularly suitable.

If an siRNA is to be administered as such, as distinct from in the form of DNA encoding the siRNA, then the strands of an siRNA can contain one or more nucleotide analogs. The nucleotide analogs are located at positions at which inhibitor activity is not substantially effected, e.g. in a region at the 5′-end and/or the 3′-end, particularly single stranded overhang regions. Preferred nucleotide analogues are sugar- or backbone-modified ribonucleotides. Nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8 position, e.g. 8-bromo guanosine; deaza nucleotides, e.g. 7-deaza-adenosine; O- and N-alkylated nucleotides, e.g. N6-methyl adenosine are also suitable. In preferred sugar-modified ribonucleotides, the 2′ OH-group is replaced by a group selected from H, OR, R, halo, SH, SR, NH2, NHR, NR2 or CN, wherein R is C1-C6 alkyl, alkenyl or alkynyl and halo is F, CI, Br or I. In preferred backbone-modified ribonucleotides the phosphoester group connecting to adjacent ribonucleotides is replaced by a modified group, e.g. of phosphothioate group. A further preferred modification is to introduce a phosphate group on the 5′ hydroxide residue of an siRNA. Such a group can be introduced by treatment of an siRNA with ATP and T4 kinase. The phosphodiester linkages of natural RNA can also be modified to include at least one of a nitrogen or sulfur heteroatom. Modifications in RNA structure can be tailored to allow specific genetic inhibition while avoiding a general panic response in some organisms which is generated by dsRNA. Likewise, bases can be modified to block the activity of adenosine deaminase.

V. Assays to Detect Modulation

Compounds are tested for their capacity to modulate expression or activity of one of the genes of the invention. Expression assays are usually performed in cell culture, but can also be performed in animal models or in an in vitro transcription/translation system. The cell culture can be of primary cells, particularly, those known or suspected to have a role in skin color, such as melancocytes or cells transfected with a gene of the invention. In the latter case, the coding portion of the gene is typically transfected with its naturally associated regulatory sequences, so as to permit expression of the gene in the transfected cell. However, the coding portion of the gene can also be operably linked to regulatory sequences from other (i.e., heterologous) genes. Optionally, the protein encoded by the gene is expressed fused to a tag or marker to facilitate its detection. The compound to be screened is introduced into the cell, usually in the form of a DNA molecule that can be expressed or directly as an RNA or protein. Expression of the gene can be detected either at the mRNA or protein level. Expression at the mRNA level can be detected by a hybridization assay, and at the protein level by an immunoassay. Detection of the protein level is facilitated by the presence of a tag. Similar screens can be performed in an animal, either natural or transgenic, or in vitro. Expression levels in the presence of a compound under test are compared with those in a control assay in the absence of compound, an increase or decrease in expression indicating that the compound modulates expression or activity of the gene.

As noted above, assays to detect modulation of a protein encoded by a gene of the invention can also be performed. In some instances, a preliminary assay is performed to detect specific binding between a compound and a protein encoded by a gene of the invention. A binding assay can be performed between the compound and a purified protein, of if the protein is expressed extracellularly, between the compound and the protein expressed from a cell. Optionally, either the compound or protein can be immobilized before or during the assay. Such an assay reduces the pool of candidate compounds for an activity assay. The nature of the activity assay depends on the activity of the gene.

Transporters can be assayed by transfecting a cell, such as an oocyte, with DNA encoding the transporter, such that the transporter is expressed in the outermembrane of the cell. The cell is then contacted with a known substrate of the transporter, optionally labeled. Uptake of the substrate can be detected by measuring intracellular label, or ionic or pH gradients across the membrane. Compounds are screened for capacity to inhibit or stimulate transport relative to a control assay lacking the substrate being tested (see WO0120331, US 2005170394, US2005170390).

Compounds that modulate expression or activity of the genes of the invention can then be tested in cell culture or animal models for modulation of skin color. The animal models can be transgenic (as described below) or nontransgenic. Compounds are tested in comparison with otherwise similar control assays except for the absence of the compound being tested. A change in skin color of the animal relative to the control indicates a compound modulates skin color.

Compounds that modulate expression or activity of the genes of the invention can also be screened in similar fashion in animal models of other diseases, particularly diseases associated in some manner with skin color. For example, the compounds can be screened in animal models of cancer, particularly skin cancer. Animal models of cancer include transgenic animals having a defect in a tumor suppressor gene (e.g., p53) or an inserted oncogene and nontransgenic animals exposed to carcinogens or into which tumor cells have been introduced. The compounds can also be screened in animal models of hypertension. A rat model of hypertension is available from Taconic Farms, German Town, N.Y.

VI. Transgenic Animals

The invention provides transgenic animals having a genome comprising a transgene comprising one of the genes of the invention, or corresponding cDNA or mini-gene nucleic acid. The coding sequence of the gene is in operable linkage with regulatory element(s) required for its expression. Such regulatory elements can include a promoter, enhancer, one or more introns, ribosome binding site, signal sequence, polyadenylation sequence, 5′ or 3′ UTR and 5′ or 3′ flanking sequences. The regulatory sequence can be from the gene being expressed or can be heterologous. If heterologous, the regulatory sequences are usually obtained from a gene known to be expressed in the intended tissue in which the gene of the invention is to be expressed (e.g., the skin).

The invention also provides transgenic animals in which a nonhuman homolog of one of the human genes of the invention is disrupted so as to reduce or eliminate its expression relative to a nontransgenic animal of the same species. Disruption can be achieved either by genetic modification of the nonhuman homolog or by functional disruption by introducing an inhibitor of expression of the gene into the nonhuman animal.

Some transgenic animals have a plurality of transgenes respectively comprising a plurality of genes of the invention. Some transgenic animals have a plurality of disrupted nonhuman homologs of genes of the invention. Some transgenic animals combine both the presence of transgenes expressing one or more genes of the invention and one or more disruptions of nonhuman homologs of other genes of the invention.

Transgenic animals of the invention are preferably rodents, such as mice or rats, or insects, such as Drosophila. Other transgenic animals such as primates, ovines, porcines, caprines and bovines can also be used. The transgene in such animals is integrated into the genome of the animal. The transgene can be integrated in single or multiple copies. Multiple copies are generally preferred for higher expression levels. In a typical transgenic animal all germline and somatic cells include the transgene in the genome with the possible exception of a few cells that have lost the transgene as a result of spontaneous mutation or rearrangement.

For some animals, such as mice and rabbits, fertilization is performed in vivo and fertilized ova are surgically removed. In other animals, particularly bovines, it is preferable to remove ova from live or slaughterhouse animals and fertilize the ova in vitro. See DeBoer et al., WO 91/08216. Methods for culturing fertilized oocytes to the pre-implantation stage are described by Gordon et al., Methods Enzymol. 101, 414 (1984); Hogan et al., Manipulation of the Mouse Embryo: A Laboratory Manual, C.S.H.L. N.Y. (1986) (mouse embryo); Hammer et al., Nature 315, 680 (1985) (rabbit and porcine embryos); Gandolfi et al. J. Reprod. Fert. 81, 23-28 (1987); Rexroad et al., J. Anim. Sci. 66, 947-953 (1988) (ovine embryos) and Eyestone et al. J. Reprod. Fert. 85, 715-720 (1989); Camous et al., J. Reprod. Fert. 72, 779-785 (1984); and Heyman et al. Theriogenology 27, 5968 (1987) (bovine embryos) (incorporated by reference in their entirety for all purposes). Sometimes pre-implantation embryos are stored frozen for a period pending implantation. Pre-implantation embryos are transferred to the oviduct of a pseudopregnant female resulting in the birth of a transgenic or chimeric animal depending upon the stage of development when the transgene is integrated. Chimeric mammals can be bred to form true germline transgenic animals.

Alternatively, transgenes can be introduced into embryonic stem cells (ES). These cells are obtained from preimplantation embryos cultured in vitro. Bradley et al., Nature 309, 255-258 (1984) (incorporated by reference in its entirety for all purposes). Transgenes can be introduced into such cells by electroporation or microinjection. ES cells are suitable for introducing transgenes at specific chromosomal locations via homologous recombination. Transformed ES cells are combined with blastocysts from a non-human animal. The ES cells colonize the embryo and in some embryos form or contribute to the germline of the resulting chimeric animal. See Jaenisch, Science, 240, 1468-1474 (1988) (incorporated by reference in its entirety for all purposes).

Alternatively, transgenic animals can be produced by methods involving nuclear transfer. Donor nuclei are obtained from cells cultured in vitro into which a human alpha synuclein transgene is introduced using conventional methods such as Ca-phosphate transfection, microinjection or lipofection. The cells are subsequently been selected or screened for the presence of a transgene or a specific integration of a transgene (see WO 98/37183 and WO 98/39416, each incorporated by reference in their entirety for all purposes). Donor nuclei are introduced into oocytes by means of fusion, induced electrically or chemically (see any one of WO 97/07669, WO 98/30683 and WO 98/39416), or by microinjection (see WO 99/37143, incorporated by reference in its entirety for all purposes). Transplanted oocytes are subsequently cultured to develop into embryos which are subsequently implanted in the oviducts of pseudopregnant female animals, resulting in birth of transgenic offspring (see any one of WO 97/07669, WO 98/30683 and WO 98/39416).

For production of transgenic animals containing two or more transgenes, the transgenes can be introduced simultaneously using the same procedure as for a single transgene. Alternatively, the transgenes can be initially introduced into separate animals and then combined into the same genome by breeding the animals. Alternatively, a first transgenic animal is produced containing one of the transgenes. A second transgene is then introduced into fertilized ova or embryonic stem cells from that animal. Optionally, transgenes whose length would otherwise exceed about 50 kb, are constructed as overlapping fragments. Such overlapping fragments are introduced into a fertilized oocyte or embryonic stem cell simultaneously and undergo homologous recombination in vivo. See Kay et al., WO 92/03917 (incorporated by reference in its entirety for all purposes).

Nonhuman homologs of human genes of the invention can be disrupted by gene targeting. Gene targeting is a method of using homologous recombination to modify a mammalian genome, can be used to introduce changes into cultured cells. By targeting a gene of interest in embryonic stem (ES) cells, these changes can be introduced into the germline of laboratory animals. The gene targeting procedure is accomplished by introducing into tissue culture cells a DNA targeting construct that has a segment that can undergo homologous recombination with a target locus and which also comprises an intended sequence modification (e.g., insertion, deletion, point mutation). The treated cells are then screened for accurate targeting to identify and isolate those which have been properly targeted. A common scheme to disrupt gene function by gene targeting in ES cells is to construct a targeting construct which is designed to undergo a homologous recombination with its chromosomal counterpart in the ES cell genome. The targeting constructs are typically arranged so that they insert additional sequences, such as a positive selection marker, into coding elements of the target gene, thereby functionally disrupting it. Similar procedures can also be performed on other cell types in combination with nuclear transfer. Nuclear transfer is particularly useful for creating knockouts in species other than mice for which ES cells may not be available Polejaeva et al., Nature 407, 86-90 (2000)). Breeding of nonhuman animals which are heterozygous for a null allele may be performed to produce nonhuman animals homozygous for said null allele, so-called “knockout” animals (Donehower et al. (1992) Nature 256: 215; Science 256: 1392, incorporated herein by reference).

VII. Methods of Polymorphic Profiling

The invention provides methods of profiling individuals at one or more SNPs of the invention. The polymorphic profile of an individual can be scored by comparison with the lighter and darker polymorphic forms occurring at each site shown in Table 1. The comparison can be performed on at least 1, 2, 5, 10, 25, 50, 100 or all 153 polymorphic sites, and optionally, others in linkage disequilibrium with them. The polymorphic sites can be analyzed in combination with other polymorphic sites. However, the total number of polymorphic sites analyzed is usually less than 1000, 100, 50 or 25.

The number of lighter and darker alleles present in a particular individual can be combined additively or as a ratio to provide an overall score for the individual's genetic propensity to lighter or darker skin color (see U.S. Ser. No. 60/566,302, filed Apr. 28, 2004, U.S. Ser. No. 60/590,534, filed Jul. 22, 2004, U.S. Ser. No. 10/956,224 filed Sep. 30, 2004, and PCT US05/07375 filed Mar. 3, 2005). Lighter skinned alleles can be arbitrarily each scored as +1 and darker skinned alleles as −1 (or vice versa). For example, if an individual is typed at all 153 polymorphic sites of the invention and is homozygous for lighter alleles at all of them, he could be assigned a score of 100% genetic propensity to lighter skin or 0% propensity to darker skin. The reverse applies if the individual is homozygous for all darker skin alleles. More typically, an individual is homozygous for lighter alleles at some loci, homozygous for darker alleles at some loci, and heterozygous for lighter/darker alleles at other loci. Such an individual's genetic propensity for skin color can be scored by assigning all lighter alleles a score of +1, and all darker alleles a score of −1 (or vice versa) and combining the scores. For example, if an individual has 102 lighter alleles and 204 darker alleles, the individual can be scored as having a 33% genetic propensity to lighter skin and 67% genetic propensity to darker skin. Alternatively, homozygous lighter alleles can be assigned a score of +1, heterozygous alleles a score of zero and homozygous darker alleles a score of −1. Thus, an individual who is homozygous for lighter alleles at 30 polymorphic sites, homozygous for darker alleles at 60 polymorphic sites, and heterozygous at the remaining 63 sites is assigned a genetic propensity of 33% for lighter skin. As a further alternative, homozygosity for alleles associated with darker skin color can be scored as 2, heterozygosity, as +1 and homozygosity for alleles associated with lighter skin color as 0.

The individual's score, and the nature of the polymorphic profile are useful in prognosis or diagnosis of an individual's susceptibility to diseases or disorders of skin color and related conditions, such as cancer, or hypertension. For example, presence of a high genetic propensity to lighter skin can be treated as a warning to avoid conditions which exacerbate the risk of cancer, such as exposure to sunlight.

Polymorphic profiling is useful, for example, in selecting compounds to modulate skin color in a given individual. Individuals having similar polymorphic profiles are likely to respond to modulators of skin color in a similar way. For example, a lighter skinned individual wishing to have a darker skin can be treated with a compound that modulates the expression or activity of a protein encoded by a gene containing a polymorphic form associated with lighter or darker skin color.

Polymorphic profiling is also useful for stratifying individuals in clinical trials of compounds being tested for capacity to modulate skin color or related conditions. Such trials are performed on treated or control populations having similar or identical polymorphic profiles (see EP99965095.5). Use of genetically matched populations eliminates or reduces variation in treatment outcome due to genetic factors, leading to a more accurate assessment of the efficacy of a potential drug.

Polymorphic profiles can also be used after the completion of a clinical trial to elucidate differences in response to a given treatment. For example, the set of polymorphisms can be used to stratify the enrolled patients into disease sub-types or classes. It is also possible to use the polymorphisms to identify subsets of patients with similar polymorphic profiles who have unusual (high or low) response to treatment or who do not respond at all (non-responders). In this way, information about the underlying genetic factors influencing response to treatment can be used in many aspects of the development of treatment (these range from the identification of new targets, through the design of new trials to product labeling and patient targeting). Additionally, the polymorphisms can be used to identify the genetic factors involved in adverse response to treatment (adverse events). For example, patients who show adverse response may have more similar polymorphic profiles than would be expected by chance. This allows the early identification and exclusion of such individuals from treatment. It also provides information that can be used to understand the biological causes of adverse events and to modify the treatment to avoid such outcomes.

Polymorphic profiles can also be used for other purposes, including paternity testing and forensic analysis as described by U.S. Pat. No. 6,525,185. In forensic analysis, the polymorphic profile from a sample at the scene of a crime is compared with that of a suspect. A match between the two is evidence that the suspect in fact committed the crime, whereas lack of a match may exclude the suspect. The present polymorphic sites can be used in such methods, as can other polymorphic sites in the human genome. However, the present polymorphic sites are particularly advantageous in that they allow prediction of certain characteristics of a suspect even before he or she is apprehended simply from the polymorphic profile of a DNA sample from the scene of the crime (see WO02/097047). For example, if the polymorphic profile of the sample indicates a high genetic propensity for lighter skin color, it can be concluded that the perpetrator is probably white. Conversely, if the polymorphic profile of the sample indicates a high genetic propensity for darker skin color, the perpetrator is probably black. Knowledge of the likely skin color of the perpetrator is useful for apprehending the right person. At this point, a sample can be taken from the suspect and compared with that of the scene of the crime, as in conventional forensic analysis.

Polymorphic profiles can be used in further association studies of traits related to skin color. Such traits include the color of other body parts, such as the hair and eyes. Such traits also include diseases, such as cancer and hypertension.

Although polymorphic profiling can be done at the level of individual polymorphic sites as described above, a more sophisticated analysis can be performed by analyzing haplotype blocks containing SNPs of the invention and/or others in linkage disequilibrium with them (see, e.g., U.S. Pat. No. 6,969,589). Each haplotype block can be characterized by two or more haplotype patterns (i.e., combinations of polymeric forms). In some instances, a haplotype pattern can be determined by detecting a single haplotype-determining polymorphic form within a haplotype block. In other instances, multiple polymorphic forms are determined within the block (see Patil et al., Science 2001 Nov. 23; 294(5547):1719-23). The haplotype pattern at each of the haplotype blocks containing SNPs of the invention in an individual is a factor in determining skin color of the individual, and can be characterized as associating with lighter or darker skin as can individual polymorphic forms. The number of haplotype blocks occupied by haplotype patterns associated with lighter skin and the number occupied by haplotype patterns associated with darker skin in a particular individual can be combined additively as for individual polymorphic forms to arrive at a percentage representing genetic propensity to lighter or darker skin. The measure is more accurate than simply combining individual polymorphic forms because it gives the same weight to haplotype blocks containing multiple polymorphic sites as haplotype blocks with a single polymorphic site. The multiple polymorphic forms within the same block are associated with the same propensity to skin color, and should not be given the same weight as multiple polymorphic forms in different haplotype blocks, which indicate independent propensity for a skin color.

The methods of the invention detect haplotypes in at least 1, 2, 5, 10, 25 50 or all of the haplotype blocks of the invention. The haplotypes can be detected in combination with haplotypes at haplotype blocks other than those of the invention. However, the number of haplotype blocks is typically fewer than 1000 and often fewer than 100 or 50.

Polymorphic forms can be detected at polymorphic sites by a variety of methods. The design and use of allele-specific probes for analyzing polymorphisms is described by e.g., Saiki et al., Nature 324, 163-166 (1986); Dattagupta, EP 235,726; Saiki, WO 89/11548. Allele-specific probes can be designed that hybridize to a segment of target DNA from one individual but do not hybridize to the corresponding segment from another individual due to the presence of different polymorphic forms in the respective segments from the two individuals.

The polymorphisms can also be identified by hybridization to nucleic acid arrays, some example of which are described by WO 95/11995 (incorporated by reference in its entirety for all purposes). Polymorphic forms can also be detected using allele-specific primers, which hybridize to a site on target DNA overlapping a polymorphism and only prime amplification of an allelic form to which the primer exhibits perfect complementarily. See Gibbs, Nucleic Acid Res. 17, 2427-2448 (1989). Polymorphic forms can also be detected by direct sequences, denaturing gradient gel electrophoresis (Erlich, ed., PCR Technology, Principles and Applications for DNA Amplification, (W. H. Freeman and Co, New York, 1992), Chapter 7), and single stranded polymorphisms analysis (Orita et al., Proc. Nat. Acad. Sci. 86, 2766-2770 (1989)). Polymorphic forms can also be detected by single-base extension methods as described by e.g., U.S. Pat. No. 5,846,710, U.S. Pat. No. 6,004,744, U.S. Pat. No. 5,888,819 and U.S. Pat. No. 5,856,092. In brief, the methods work by hybridizing a primer that is complementary to a target sequence such that the 3′ end of the primer is immediately adjacent to but does not span a site of potential variation in the target sequence. That is, the primer comprises a subsequence from the complement of a target polynucleotide terminating at the base that is immediately adjacent and 5′ to the polymorphic site. The hybridization is performed in the presence of one or more labeled nucleotides complementary to base(s) that may occupy the site of potential variation. Some polymorphic forms resulting in a corresponding change in encoded proteins can also be detected at the protein level by immunoassay using antibodies known to be specific for particular variants, or by direct peptide sequencing.

VIII. Expression Monitoring

The invention also provides methods of expression profiling by determining levels of expression of one or more genes shown in Table 3. The methods preferably determine expression levels of at least 2, 5, 10, 15, 20, 25, 29, 50, 100 or all of the genes shown in Table 3. Preferably, the expression levels are determined of at least 2, 5, 10, 15, 20, 25 or all 29 of the genes containing polymorphic sites of the invention. Optionally, expression levels of other genes beyond those associated with skin color in the present application are also determined. However, the expression profile is preferably not determined at more than 1000, 5000, or 10,000 genes.

The expression levels of one or more genes in a discrete sample (e.g., from a particular individual or cell line) are referred to as an expression profile. Typically, the expression profile is compared with an expression profile of the same genes in a control sample. For example, the expression profile in an individual with relatively darker skin can be compared with the expression profile of an individual with relatively light skin to determine genes that are differentially expressed between the two skin types. The individuals with relatively dark and relatively light skin can be selected from the upper and lower quintiles of the same race or from different races (e.g., African and Northern European respectively). Expression levels can also be compared with an individual having cancer of other disease of the skin with a normal control to identify genes differentially expressed in a disease state. The controls can be contemporaneous or historical. Individual expression levels in both the test and control samples can be normalized before comparison, e.g., by reference to the level.

Gene expression profiles can also be compared in skin cells exposed to a known skin toxin relative to control. These gene expression profiles are useful in characterizing whether a test compound is a toxin. The skin cells are exposed to the test compound, and the gene expression profile determined. The gene expression profile is then compared to a gene expression profile of skin cells exposed to a known toxin or a control. If the gene expression profile in the presence of the test compound is more similar to that in the presence of the toxin than that in the presence of the control, one can conclude that the test compound is likely to be a toxin. Conversely, if the gene expression profile of the test compound is more similar to that in the presence of the control than that in the presence of the toxin, one can conclude that the test compound is likely not toxic or at least less toxic than the known toxin.

Knowledge of which genes are differentially expressed with different skin color is useful for selecting appropriate compounds to modulate skin color. For example, if a gene is more highly expressed in darker skin than lighter skin, a compound that decreases expression of the gene or activity of the gene product is useful to lighten color. Conversely, a compound that increases expression of the gene or activity of the gene product is useful to darken skin color. Similarly, if a expression of a gene is elevated in a disease state relative to a normal individual, then a compound that decreases expression of the gene or activity of the gene product may be useful to treat the disease.

IX. Variant Proteins

Some of the polymorphic sites of the invention are characterized by presence of polymorphic forms encoding different amino acids. Such polymorphisms are referred to as non-synonymous indicating that the different polymorphic forms are translated into different protein variants. The invention further provides such variant proteins or fragments thereof in isolated form. In some embodiments, the variant proteins or fragment thereof retain the activity of the full length protein. Example of variants proteins include a protein encoded by SLC24A5 with one or the other of the polymorphic forms at position 46213776, and a protein encoded by ATP8B4 with one or the other of the polymorphic forms at position 48013605.

IX. Methods of Treatment

Compounds having activity in modulating expression or activity of a gene of the invention can be used in methods of modulating skin color. These methods can be performed for cosmetic purposes to lighten or darken skin color to a desired hue. The methods can also be used in prophylaxis or treatment of various diseases and conditions associated with skin color.

Diseases and disorders associated with skin color are broadly classified as hyper and hypo pigmentation. Hyper pigmentation is a common, usually harmless condition in which patches of skin become darker in color than the normal surrounding skin. This darkening occurs when an excess of melanin, the brown pigment that produces normal skin color, forms deposits in the skin. Hyper pigmentation can affect the skin color of people of any race. Age or “liver” spots are a common form of hyper pigmentation. They occur due to sun damage, and are referred to as solar lentigines. These small, darkened patches are usually found on the hands and face or other areas frequently exposed to the sun. Melasma or chloasma spots are similar in appearance to age spots but are larger areas of darkened skin that appear most often as a result of hormonal changes. Pregnancy, for example, can trigger overproduction of melanin that causes darkened skin on the face, abdomen and other areas. Women who take birth control pills may also develop hyper pigmentation because their bodies undergo similar kind of hormonal changes that occur during pregnancy. Hyper pigmentation is also seen in cases of hyperpituitarism and Addison's disease. Hyper pigmentation can also result from skin diseases, such as acne or injuries to the skin, including some caused by surgery.

Hypo pigmentation falls into several categories: albinism, disease-related hypo pigmentation, injury-related hypo pigmentation, vitiligo, drug- and chemical-related hypo pigmentation. People who are genetically unable to produce melanin are called albinos. Skin cancers are common in albinos who live in sunny climes. Many inflammatory disorders, such as psoriasis, result in a temporary pigment loss in the skin. Traumatic injuries (such as bums or freezing) also cause loss of pigmentation through destruction of melanocytes. Vitiligo causes the loss of pigment in areas of skin probably due to an autoimmune attack on melanocytes. In all races, vitiligo is the major cause of acquired, widespread pigment loss. Vitiligo can occur at any age; however, about 50% of the cases appear between the ages of 10 and 30 years. The incidence of vitiligo is higher in females than in males. Certain drugs and chemicals can also cause hypo pigmentation.

Some examples of particular diseases of the skin include Hermansky Pudlak syndrome types 1 through 7, pigment-dispersion syndrome (GPDS1), oculocutaneous albinism type 1 (OCA1), oculocutaneous albinism type 3 (OCA3), oculocutaneous albinism type 4 (OCA4) ocular albinism type 1, Griscelli syndrome, Usher syndrome type IB, Chediak Higashi syndrome, autosomal recessive osteopetrosis, xeroderma pigmentosum group D, Ectodermal dysplasia type 1, Waardenburg-Shah Syndrome, Hirschsprung's disease type 2, Familial incontinentia pigmenti (IP), Waardenburg syndrome type 1, Waardenburg syndrome type 2, Waardenburg syndrome type 3, Piebaldism, skin cancer, and melanoma.

Compounds of the invention can be used in combination with other skin treatments. Existing treatments include creams used to lighten the skin. Most contain hydroquinone, which bleaches, lightens, and fades darkened skin patches by slowing the production of melanin so those dark spots gradually fade to match normal skin coloration. In more severe cases prescription creams with tretinoin and a cortisone cream are used. Laser treatments can also be used.

A compound can be administered to a patient for prophylactic and/or therapeutic treatments. A therapeutic amount is an amount sufficient to remedy a disease state or symptoms, or otherwise prevent, hinder, retard, or reverse the progression of disease or any other undesirable symptoms in any way whatsoever. In prophylactic applications, a compound is administered to a patient susceptible to or otherwise at risk of a particular disease or infection. Hence, a “prophylactically effective” amount is an amount sufficient to prevent, hinder or retard a disease state or its symptoms. In either instance, the precise amount of compound contained in the composition depends on the patient's state of health and weight.

An appropriate dosage of the pharmaceutical composition is determined, for example, using animal studies (e.g., mice, rats) to determine the maximal tolerable dose of the bioactive agent per kilogram of weight. In general, at least one of the animal species tested is mammalian. The results from the animal studies can be extrapolated to determine doses for use in other species, such as humans for example.

The pharmaceutical compositions can be administered in a variety of different ways. Compositions are often administered as creams, lotions or emoluments onto the skin. Compounds can also be administered as a composition containing a pharmaceutically acceptable carrier via oral, intranasal, rectal, topical, intraperitoneal, intravenous, intramuscular, subcutaneous, subdermal, transdermal, intrathecal, and intracranial methods. The route of administration depends in part on the chemical composition of the active compound and any carriers.

For administration to the skin, a composition used according to the invention also comprises a dermatologically/cosmetically acceptable vehicle to act as a dilutant, dispersant or carrier for the actives. The vehicle can comprise materials commonly employed in skin care products such as water, liquid or solid emollients, silicone oils, emulsifiers, solvents, humectants, thickeners, powders, propellants and the like.

The vehicle usually forms from 5% to 99.9%, preferably from 25% to 80% by weight of the composition, and can, in the absence of other cosmetic adjuncts, form the balance of the composition.

Besides the actives, other specific skin-benefit actives such as sunscreens, skin-lightening agents, skin tanning agents can also be included. The vehicle can also include adjuncts such as antioxidants, perfumes, opacifiers, preservatives, colorants and buffers.

Topical composition used in the method of the present invention can be prepared by conventional methods for preparing skin care products. The active components are generally incorporated in a dermatologically/cosmetically acceptable carrier in conventional manner. The active components can suitably first be dissolved or dispersed in a portion of the water or another solvent or liquid to be incorporated in the composition. The preferred compositions are oil-in-water or water-in-oil or water-in-oil-in-water emulsions.

The composition can be in the form of conventional skin-care products such as a cream, gel or lotion, capsules or the like. The composition can also be in the form of a so-called “wash-off” product e.g. a bath or shower gel, possibly containing a delivery system for the actives to promote adherence to the skin during rinsing. Most preferably the product is a “leave-on” product; a product to be applied to the skin without a deliberate rinsing step soon after its application to the skin.

The composition can be packaged in any suitable manner such as in ajar, a bottle, tube, roll-ball, or the like, in the conventional manner.

The method of the present invention can be carried out one or more times daily to the skin which requires treatment. The skin benefit usually becomes visible after 1 to 6 months, depending on skin condition, the concentration of the active components used in the inventive method, the amount of composition used and the frequency with which it is applied. In general, a small quantity of the composition, for example from 0.1 to 5 ml is applied to the skin from a suitable container or applicator and spread over and/or rubbed into the skin using the hands or fingers or a suitable device. A rinsing step may optionally follow depending on whether the composition is formulated as a “leave-on” or a “rinse-off” product.

The components of pharmaceutical compositions are preferably of high purity and are substantially free of potentially harmful contaminants (e.g., at least National Food (NF) grade, generally at least analytical grade, and more typically at least pharmaceutical grade). To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions. Compositions for oral administration need not be sterile or substantially isotonic but are usually made under GMP conditions.

IX. Other Uses of Polymorphisms

Polymorphisms of the invention are also useful in screening individuals for presence or susceptibility to diseases affecting skin color, or associated with skin color, such as cancer and hypertension. Polymorphic forms or haplotype patterns associated with skin color can be a risk factor for cancer or a protective factor against cancer and/or hypertension. For example, polymorphic forms or haplotype patterns associated with darker skin may be a risk factor for hypertension and/or a protective factor against cancer. If an individual is screened for polymorphic forms or haplotypes at a plurality of sites or haplotype blocks, the risk factors and protective factors against a given disease can be combined to give an overall factor representative of risk of or protection from disease. For example, if a subject has 20 risk factors of disease, and ten protective factors against the disease, the individual could be assigned an overall risk factor of 10. After prognosis or diagnosis of such a disease, the individual is informed of the prognosis or diagnosis and counsel to take remedial measures. These can include avoiding sunlight to lessen risk of cancer and avoiding salt, smoking, lack of exercise and stress to reduce risk of hypertension. These can also include administration of therapeutics, for example, to prevent the development of hypertension in an at-risk individual.

Polymorphic forms can also be further characterized for their effect on the activity of a gene or its expression levels. Polymorphic forms occurring within a protein coding sequence are likely to effect activity of the encoded protein particularly if the change between forms is nonsynonymous. Polymorphic forms occurring between genes are more likely to affect expression levels. Polymorphic forms occurring in introns can affect expression levels or splice variation.

Compounds that modulate skin color are likewise useful for treatment or prophylaxis of cancer or hypertension. Compounds that increase skin color are useful for treatment or prophylaxis of cancer, and compounds that decrease skin color are useful for treatment or prophylaxis of hypertension.

### EXAMPLES

1. Association Studies

An association study was performed on populations of darker and lighter skin-colored volunteers having ancestral origin in the subcontinent of India (India, Pakistan, Bangladesh, Sri Lanka). These two populations are sometimes collectively referred to as the original populations. The “darker” and “lighter” skin-colored volunteers were each identified from the estimated top and bottom 20% of the total distribution of skin color for a South Asian population sample. All individuals included in the study were assessed for population stratification (“matched”) by individually genotyping each with a random genomic set of >300 SNPs to reduce the number of false positive associations (see US 20040220750). Determining associations between populations of different skin color within a given geographic population sample reduces the risk of selecting polymorphisms that discriminate between ethnicity unrelated to skin color.

Volunteers were recruited in the UK, who were able to confirm the ancestry of all 4 of their grandparents from the sub-continent of India (India, Sri Lanka, Pakistan, Bangladesh). Ethical approval for the study was obtained from an appropriately constituted review board and informed consent was obtained from all volunteers. The intrinsic skin color of the volunteers was measured using a Minolta chromameter and a 30 ml blood sample taken for subsequent DNA isolation. One chromameter output is annotated ‘L*’ which indicates the reflectance of the [skin] surface from which the measurement was taken. L* was measured on six sites on each volunteer, 3 from each arm, with 2 reflectance readings per arm from sun-protected sites and one value from a sun exposed site. The highest L* value (i.e. the L* value indicating the highest reflectance and therefore lightest color of the skin surface) was taken as the measure of natural (intrinsic) skin color of the volunteer.

Using the L* values a sample distribution of intrinsic skin color was determined and the estimated 20% tails of the sample distribution was calculated. After the color phenotype of the sample distribution had been determined, it was found possible to enhance the recruitment and sampling of volunteers having the required relative lighter or darker skin color. Overall the boundary for the lighter tail of the distribution was found to be for L* values greater than 63 and the darker tail of the distribution was found to be for L* values of less than 56. In total the intrinsic skin color of more than 3000 unrelated volunteers was measured at over 50 recruitment sites in the UK.

DNA was purified from the blood sample using standard commercially available kits (e.g. as supplied by Quiagen), from 1171 volunteers of which 923 had intrinsic skin color falling into the lighter or darker 20% tails of the population distribution.

The L* value from the chromameter reading is highly correlated with the amount of melanin present in skin (Alaluf et al., Pigment Cell Research 15 (2), 119-126 (2002)). Accordingly a genotyping study of human populations separated by L* value investigates the SNPs associated with melanin level modulation and skin color modulation in vivo.

DNA from volunteers in either the “lighter” or “darker” distribution of the population sample was pooled. The two pools of lighter and darker distributions were genotyped at 1.3 million SNPs distributed throughout the genome (see US 20040029161 and U.S. Ser. No. 10/970,761, filed Oct. 20, 2004 for discussion of genotyping pooled populations). Genotyping was performed using GeneChip® arrays from Affymetrix, as described in US 20040029161). The top 30,000 SNPs with the largest estimated allele frequency difference between the “lighter” and the “darker” populations were chosen for further investigation. All of these SNPs had an allele frequency difference of at least 10% and a p-value less than 0.01.

Each of the volunteers in the initial populations was individually genotyped at each of the top 30,000 polymorphisms. Genotyping was performed with a GeneChip® array containing probes customized to these polymorphisms. The top polymorphisms with the largest allele frequency difference between lighter and darker volunteers were selected for a validation study.

The validation study analyzed the polymorphisms from the previous study and some additional polymorphisms on new sample populations comprising 116 volunteers with ancestral origin from the subcontinent of India having either “lighter” or “darker” skin color as defined above. These two populations are sometimes referred to as replicate populations.

The results from the study on the original populations (set 1) and replicate populations (set 2) are summarized in Table 4 (D=darker colored skin and L=lighter colored skin). The first two columns show the chromosome number and polymorphic site position as in other tables. The third column shows the reference allele whose frequencies are reported in the subsequent “Reference Allele Frequency” columns. The reference allele may be associated with lighter or darker skin depending on the polymorphic site. Columns 4-7 provide data from analysis of set (1). Columns 4 and 5 provide the allele frequencies of the reference allele in lighter- and darker-skinned volunteers. Column 6 gives the p-value for the association test. The association test was corrected for population structure in the sample set using the Genomic Control correction (Bacanu, Am. J. Humn. Genet. 66, 1933-1944 (2000). Column 7 gives the False Discovery Rate. This is the estimated fraction of false positives at a given level of significance in the data (Storey et al. PNAS 100, 9440-9445 (2003)). Columns 8-11 provide similar information for set 2 of the replicate populations. Columns 12-14 provide information from combined analysis of the first and second set (when performed). Column 12 is the difference in allele frequency (darker-skinned volunteers minus lighter-skinned volunteers) and column 13 and 14 are the p-value for the association test and false discovery rate, as before.

The combined populations identified 153 polymorphisms associated with variation in human skin color. The criteria for identification of these were as follows. For SNPs genotyped in both sample sets 1 and 2, SNPs were included if (a) the false discovery rate in the joint analysis was ≦0.01 or (b) (allele frequency in darker-skinned volunteers minus allele frequency in lighter-skinned volunteers) ≧0.10 in sample set 1 and (allele frequency in darker-skinned volunteers minus allele frequency in lighter-skinned volunteers) ≧0.09 in sample set 2. For SNPs genotyped only in sample set 2, SNPs were included if (a) the false discovery rate was ≦0.1 in sample set 2 or (allele frequency in darker-skinned volunteers minus allele frequency in lighter-skinned volunteers) ≧0.09 in sample set 2.

119 of the previously identified 153 SNPs were also genotyped on 24 volunteers of two populations: African Americans (AA), and European Americans (EA). For each of these 119 SNPs, the delta-p between the European Americans and the African-Americans was calculated (AA-EA) and compared to the overall delta-p between the lighter skinned volunteers and darker skinned volunteers from the skin pigmentation study (D-L). The data are shown in Table 5. Columns 1 and 2 show the chromosome number and polymorphic site position as in other tables. Column 3 shows the identity of the reference allele used for comparison. Column 4 shows the difference in frequency of the reference allele between darker and lighter skinned South Asians. Column 5 shows the difference in frequency of the reference allele between AA and EA. Although the exact skin color of the volunteers in the American sample set was not known, it can be assumed that the European Americans are of fair skin and the African Americans are of a darker hue. If the delta-p's of both population sets are in the same direction (−/+), then the SNP was considered to show consistent allele frequency differences in the two population sets relative to skin pigmentation. A failure to give a consistent delta-p in the American population compared to the South Asian population is not evidence that the association with skin pigmentation in the South Asian population is false, but a consistent correlation between the delta-p in the two populations sets does support the theory that the association between skin pigmentation and the SNP is present in multiple ethnically diverse populations. Most polymorphisms identified in the South Asian study gave delta-p's in the same direction between darker and lighter skinned South Asians, and between African Americans and European Americans

2. Determination of Gene Expression (RNA Levels) in Human Skin and Human Skin Derived Melanocytes.

The majority of genes found to be associated with human skin color are expected to be expressed in skin. ‘Expressed’ means that the gene is translated into detectable RNA. A gene that is expressed in skin but not in cultured melanocytes is presumed to be expressed in other skin cell types such as the dermal fibroblast or keratinocyte. These other cell types also contribute to melanocyte function in skin. For example skin color is strongly influenced by the control of the transfer of melanosomes from melanocytes to keratinocytes. Skin color is also affected by keratinocyte function, such as by the regulation of melanosome distribution inside keratinocytes and by the degradation of melanosomes by keratinocytes. A gene that is neither expressed in skin nor melanocytes can still influence skin color by a systemic route. Melanocyte stimulating hormone (MSH) is an example of a protein acting by such a mechanism.

Punch biopsies of skin 4 mm in diameter were removed from the upper inner forearm of study volunteers, a site representing the intrinsic skin color and biology of the volunteer, unaffected by sun exposure. RNA transcripts in the tissue were stabilized by placing the skin biopsies immediately in ‘RNA-later’ RNA stabilization reagent purchased from Qiagen and storing the biopsies at −20° C.

RNA extraction was performed using a Qiagen ‘RNeasy’ kit. Skin biopsies were chopped into small pieces with a scalpel and placed into the lysis buffer supplied with the kit; the buffer supplemented with freshly added 15 mM dithiothreitol (DTT). The lysate was disintegrated using a rotor-stator homogenizer for 60 seconds and extracted using phenol chloroform/chloroform phase separation. The resulting supernatant was mixed with 70% ethanol as described in the RNeasy kit protocol and purified according to the protocol, including on-column DNase treatment. The purified RNA eluate was quantified using an Agilent Bioanalyser and stored in aliquots at −80° C. cDNA was synthesized using the Roche AMV 1st strand cDNA synthesis kit.

Primary melanocytes were derived from donor foreskins using standard methods and cultured in medium 254CF (purchased from Cascade Biologics; www.cascadebio.com), supplemented with 0.2 mM calcium chloride solution and 100 fold diluted human melanocyte growth supplement (also purchased from Cascade Biologics). Human melanocyte cultures may also be obtained commercially (e.g. also from Cascade Biologics). RNA containing lysates were prepared from the melanocytes using RNA lysis buffer purchased from Ambion Inc. (http://www.ambion.com). RNA was purified using the Ambion Inc. RNaqueous kit protocol. Globally amplified PolyAcDNA was prepared from 200 ng total RNA as described by Bardy and Iscove in Methods in Enzymology 1993, vol 225, pages 611-623.

Real-time quantitative polymerase chain reaction (qPCR) was used to quantify RNA levels, using the cDNA as a template using a BioRad iCycler and Biorad SYBR® Green reaction mix. RNA levels were normalized to GAPDH expression. The primer human DNA sequences used in the qPCR reactions are listed below:

| | |
|---|---|
| 5′ CATGCCTCCTCACTACCGCTAC 3′ | for MATP |
| 5′ ATCTGTGAAGAACAGCATGTTGGAC 3′ | |
| 5′ TCATGCTGAACAGACTCGCAGG 3′ | for MATP |
| 5′ TCCATCCAATGAGGTGGCTGATG 3′ | (crosses exons) |
| 5′ CCTTGGATTGTCTCAGGATGTTGC 3′ | for SLC24A5 |
| 5′ GGATGGTGCTAATGCCAATATCTCC 3′ | |
| 5′ GACCTGCTCTCCTGGACATAACTC 3′ | for SLC12A1 |
| 5′ CCATGCCACTGTTCATCTCCTTAAC 3′ | |
| 5′ GGAAGATGATCAAGCTGGTGTTGTG 3′ | |
| 5′ AATCCAGGAGAGGCGAATGAAGAG 3′ | |
| 5′ ACAGCCTATTAGTGCCAGCCAG 3′ | for MYEF2 |
| 5′ GCTATTCATTGCTTCCAGACCACC 3′ | |
| 5′ CAGTCTGAAGTGCTCATCAACAGC 3′ | for ATP8B4 |
| 5′ AGAGACCATGTGGCTCACTACTTG 3′ | |
| 5′ CCACGGTCAGGCTTGGCTG 3′ | for DUT |
| 5′ AATGAGCTGTGCAATTCGATCACC 3′ | |
| 5′ TGAAGGCAAGGTGAGGACCAAG 3′ | for SHC4 |
| 5′ TAAGGCTTACTTCGCTTCCAGAGG 3′ | (formerly RALP) |
| 5′ GGCATGACGGTGAGAGGTCTG 3′ | for GRM5 |
| 5′ TCTGTCACATCATACCTGTCAGCC 3′ | |
| 5′ CCTGGACTATCTGCTGGAGATGC 3′ | for DRG2 |
| 5′ TGATGGCGTCTGTGAAGTCTGG 3′ | |
| 5′ GAGCAGATAGACTGGCAGGAGATC 3′ | for MYO 15a |
| 5′ TGGCACTTCTGTAGGAAGGTGTG 3′ | |
| AGCCGCTCTTGAAGAAGCCG 3′ | for CRI-1 |
| 5′ GTCAGACGATTGACAACCATCAGTG 3′ | |
| 5′ ATCACCTGTACCTGGATGAAGTTCC 3′ | for MYLK |
| 5′ CTTGCTGCCATTCTCGCTGTTC 3′ | |
| 5′ GGATGGTGTTGCCTCTCCTCG 3′ | for ALK |
| 5′ ATCTTGTCCTCTCCGCTAATGGTG 3′ | |
| 5′ GCTTATCCAGATCACTTCAGCATCG 3′ | for DDB1 |
| 5′ CTGCCTACAGCCACCACCAC 3′ | |
| 5′ GCTGGTGGTGAGTGTATTAACAACC 3′ | for FBN1 |
| 5′ CTCATCAATGTCTCGGCATTCTGTC 3′ | |
| 5′ CGACTGGAAATGCTTTACGGAAG 3′ | for Sema6D |
| 5′ CGTAACACATCTCAGCACCGA 3′ | |

Table 6 below summarizes the results for 18 genes having the highest allele frequency difference, associated with natural variation in skin color. If no primer sequences are provided, expression information comes from publications in the scientific literature (genes TYR and OCA2).

TABLE 6

| Gene name | Expressed in skin? | Expressed in Melanocytes |
|---|---|---|
| SLC24A5 | Yes | Yes |
| MYEF2 | Yes | Yes |
| SLC12A1 | No | No |
| DUT | Yes | Yes |
| MATP | Yes | Yes |
| FBN1 | Yes | No |
| SEMA6D | Yes | Not determined |
| ATP8B4 | Yes | Yes |
| TYR | YES | Yes |
| OCA2 | Yes | Yes |
| DRG2 | Yes | Yes |
| MYO15A | No | No |
| GRM5 | Yes | Yes |
| DDB1 | Yes | Yes |
| SHC4 (RALP) | Yes | Yes |
| CR1 | Yes | Yes |
| ALK | No | No |
| MYLK | Yes | Not determined |

The gene product of genes expressed in melanocytes can directly affect melanocyte pigment production or the transfer of pigment from melanocytes to keratinocytes. Data from this study show melanocyte-specific expression of SLC24A5 in skin providing support for a role of this gene in regulation of skin color. The transcript levels for MATP and MYEF2 were higher in skin biopsies derived from volunteers with darker skin color compared to volunteers with lighter skin color. This result suggests that the expression of these genes and subsequent manufacture of greater levels of protein in dark melanocytes is one mechanism by which one or both of these genes regulate skin color. These genes may also regulate skin color by other biological mechanisms.

Various embodiments and modifications can be made to the invention disclosed in this application without departing from the scope and spirit of the invention. Unless otherwise apparent from the context any embodiment, feature or element of the invention can be used in combination with any other. All patent filings and publications mentioned herein are incorporated by reference for all purposes to the same extent as if each were so individually denoted.

TABLE 1

| Genomic Location | | dbSNP annotation | | | | |
|---|---|---|---|---|---|---|
| (NCBI Build 35) | | Perlegen | | Darker | Lighter | SNP Assay with ambiguity code at SNP |
| Chr | Position | SS_ID | RS_ID | Allele | Allele | location |
| 15 | 46179457 | 23427569 | 1834640 | G | A | ACCTCAGAAACCACRACATAAACCAAGGA |
| 15 | 46275146 | 46552216 | 12913316 | C | T | ACTCAGTTCAAATAYAATCTCTTGCAAGA |
| 15 | 46258816 | 23997762 | 11070627 | A | T | AAAGTAATACTCAAWTAACATAATTTCAT |
| 15 | 46056053 | 23426241 | 2924566 | G | A | CCATTCCTGGGGATRAGAAGCCAGTAACA |
| 15 | 46420445 | 24441261 | 11637235 | C | T | TTTAAAACCCAAATYGTAATTTTCTCCTA |
| 15 | 46098702 | 23426809 | 9788730 | C | A | TTCCCCAATTCACTMCCTGCTCAGACTGT |
| 15 | 46087470 | 23426669 | 4775730 | C | T | GCAAAGTAGAGGAGYAGATGGATCAGGAA |
| 15 | 46473467 | 23998787 | 10519170 | G | A | TGATTTCTCCATTCRTTGCTTGGCTCTTA |
| 15 | 46049012 | 23996602 | 2965317 | C | T | ACCATTCCATGTTAYGGTGTTTCTGCCAA |
| 15 | 46097633 | 23996961 | 7164700 | A | G | ATTTGGTTGCATCCRACACCAGGCAAGGG |
| 15 | 46051787 | 23996619 | 2965318 | T | G | CAAAAACCCATTCAKATTCAAGGGATTAT |
| 15 | 46472393 | 23998780 | 1820489 | C | T | CTCTTCGCCCTCTCYGGGGATGTTCGGGT |
| 15 | 46306954 | 23997906 | 16960682 | C | G | TTCTTTGTACCTTGSATGAGACCCACTGG |
| 15 | 46157395 | 23427291 | 16960541 | T | G | ATTACGGTCATGATKAACTGAAACCCTTA |
| 15 | 46971973 | 24444943 | 4774527 | G | A | GGATAACACAGATARTTGGGCCCTCTGGC |
| 5 | 33987450 | 23456916 | 16891982 | C | G | ACGGAGTTGATGCASAAGCCCCAACATCC |
| 15 | 46986684 | 24445025 | 11854994 | G | A | TGATAACGGTCATGRTGATGTGTGATTTC |
| 15 | 46313654 | 46552215 | 2413890 | G | T | GTTGATTGTTTATGKTATTTATGCATGTG |
| 15 | 45957669 | 23996108 | 504376 | C | G | GCCTGACCTTGAATSAAGCCATTTATTCT |
| 15 | 46861195 | 24000770 | 7176696 | G | A | GGTTTGCCAAGAACRGGTTGTACTTTAGC |
| 15 | 46843962 | 24000655 | 8041414 | G | T | ACTTGTTGTGCGTGKCTTGGATAGCAAAA |
| 15 | 46827089 | 23432830 | 784411 | C | T | TGAATCCTAAAGGAYGAGAGTAAGACTAA |
| 11 | 88551344 | 24427553 | 1042602 | C | A | ACTGCTTGGGGGATMTGAAATCTGGAGAG |
| 15 | 46521916 | 23999088 | 10519174 | G | A | TCATAGAAGATGACRCTCCTGATTTGTGG |
| 15 | 46039330 | 23996511 | 2924572 | T | A | CTCCCTAGAGTAGAWTGTGGTTTGAGAGA |
| 15 | 47018806 | 24001819 | 4592603 | G | A | GTCAGAGGAAGGACRCTGGGGCGAGTTA |
| 15 | 46055778 | 24439241 | 2924567 | G | T | TGTGTGCCCAAGAAKAAAGGGTAAACACT |
| 15 | 46157464 | | | G | A | TTCTGGGGGTGTTARTTTTGCTGAGTAGG |
| 15 | 46930524 | 24444678 | 17467239 | G | T | ATATGCAACATTCTKGGCCTATCTGAGAA |
| 15 | 47009173 | 24445254 | 17384518 | G | A | AACAGCAGATGTGARTCCAAACTGCTCTG |
| 15 | 46979618 | 24001531 | 4775785 | C | T | AATTTTGTTTTCAAYGTAGTCACTCTATA |
| 15 | 46637949 | | 12441775 | C | G | GAAGCATAAATTATSTAAGTCATCTTACA |
| 15 | 46569855 | 24442308 | 11635140 | C | T | GAGATCCCACAGTGYTCTTTCGGGAGATG |
| 15 | 46963495 | 24001439 | 4775783 | T | A | CTATGTTCTTTGCAWCTTAGTTCUCATT |
| 15 | 46731907 | 24128976 | 7162626 | C | T | TCACCCAGGGACCCYATCCACAAAATGCA |
| 15 | 45903689 | 23995620 | 494230 | C | T | TGCCCATGTGCACAYCAAGGTAGACAAAC |
| 15 | 45922856 | 24438306 | 785016 | T | C | AAAAGTCATTGTTGYTAAAGCGGGTCAAC |
| 15 | 46791064 | 24443744 | 17463995 | T | C | TCAATCCCTTTAGCYGTTTTCTAGTATTT |
| 2 | 126436772 | 23212788 | 730251 | T | G | GACCCATTGACTAAKAAACATTTTTGTTG |
| 15 | 45965538 | 23996156 | 491996 | A | T | TCTGGGGAAGGGAAWTGGCATTGGAACAT |
| 2 | 126436342 | 23212756 | 11685174 | A | G | GGGACCATCTACAARCATTATTTTTTTAA |
| 15 | 46892226 | 24444404 | 7182710 | A | T | TTTACAGATTGGTAWATTCTTTCACAAGC |
| 15 | 46089356 | 24439565 | 17423970 | G | A | GATGCATACTAAGTRAGGGGAGAGTTCTA |
| 15 | 45970045 | 23996178 | 677207 | C | G | TTGGGATGGGAGAASAGCTGCCAAGTCAG |
| 15 | 46800217 | 24000333 | 784416 | G | C | ATTGATTATTCTCTSTGCTGCACCTATAT |
| 2 | 126433598 | 23212684 | 1869746 | A | G | TATTTGTGTGGGAARAGCTTTCAAAGCCT |
| 18 | 36327583 | 24488140 | 1991885 | G | A | ATATTCCCTTAATCRGAAAAGAGAGTGAC |
| 15 | 46918903 | 24444591 | 4775777 | A | G | TTTCTCCAACATCTRCTTTAAGTATGCAC |
| 7 | 19893304 | 23705911 | 6461477 | A | G | TCACTACAAAAACARTATGAATATGATAC |
| 15 | 44862136 | 46552227 | 1918641 | A | G | TTAACGTTTTTCTRCCACAATTGCTACA |
| 14 | 23885591 | 24709550 | 4981507 | A | G | TTGCTGTGTTTCCARTATGAAGAACATAT |
| 15 | 25671898 | 24188884 | 3893201 | C | T | AGGGGAATTTAAAAYGTCCTAGGCCAATG |
| X | 109181255 | 23827871 | 5942629 | A | G | GTCTCAGTTTGAAGRAGTGATAAATAAAT |
| 11 | 88159190 | 24406643 | 12802000 | C | T | AAGAATTCTTAATGYATTGCTTTGCCATG |
| 11 | 88154670 | | 7119749 | A | G | CCATGCTGAGCAGARGAATTACAAGCAAT |
| 15 | 46034246 | 24438996 | 751467 | G | A | AACATATATGTGTARAGCAAAAATATTTT |
| 15 | 46834784 | 24000568 | 1699400 | G | A | ATTTGCTTGTTTCTRTATCAATACCTTTG |
| 5 | 172955019 | 23362437 | 421239 | A | G | AGAAGTGATTTTCCRGCGAGAAGCAGCGG |
| 14 | 82033421 | 24110571 | 10134177 | G | T | ATACCAATAATCATKTATGATACACTTTC |
| 15 | 45944278 | 23425298 | 669653 | A | G | ACAAAGGTGCTTACRTTGTGAATAATGAC |
| 8 | 17415668 | 23734807 | 17124738 | G | T | TTGACCAAGCAAAAKTGACTTTTTGTCCC |
| 15 | 46837115 | 24000579 | 1968825 | C | T | GTCAAAAGACAGAAYTGGGCATCTCCAAA |
| 14 | 82039583 | 24110602 | 8006130 | A | G | TGTGAGAGACTGAGRATAAGCAGAAAAGG |
| 11 | 88208027 | 24407415 | 492312 | A | G | TGGAAATGTCTTACRTGATAAACCTGATA |
| 16 | 4154994 | 24217179 | 11640791 | T | A | GTTTTGCGACTCCAWACTGATCACCGTTG |
| 15 | 44942842 | 23989034 | 8031322 | C | T | CTAATTTCTGTCACYGGACTTAAATTCAG |
| X | 118758045 | 46556429 | 6646491 | A | T | AAATAGGCTTGTACWATCCATCTATTAAT |
| 15 | 46974966 | 24001507 | 7162426 | A | G | TCGTTAATACCCGCRTGGCTGGTAAACTA |
| 3 | 125017560 | 24334272 | 13094938 | C | T | TACAAACCCAGGTCYTGCTCATAGGCATT |
| X | 118748996 | 23831607 | 4825677 | T | C | CGTTGTGTTCACTAYCATAGTGTCAGTGC |
| 11 | 88193818 | 24407163 | 7479952 | T | C | ATAAGACATCATTTYAGAAATATATACAA |
| 15 | 44939404 | 23988996 | 11070543 | T | C | TTGTATAACAGAGCYATAAGAAATAAGAC |
| 15 | 58988635 | 24718848 | 1054789 | T | A | AATTCTTGCTGTGGWCCCAGCGGTGAGCA |
| X | 33404293 | 24234257 | 2860053 | C | T | GAAACATCTGAkAAYCAAATTATCAAAGT |
| 2 | 76405736 | 23881131 | 10496203 | G | T | TTAATTTATACTACKTCTAGAAACAAACA |
| 2 | 209313998 | 14888882 | 10497903 | C | T | AAAGTGTTCTTCAAYATTCATACTACTTT |
| 6 | 48642656 | 24145831 | 16877564 | C | G | TCCCAGTCAAGGCASGTAGGATCCCTATT |
| 18 | 59141498 | | 1944423 | A | G | TCATCAATATAAATRTTCTCCAAGTTTAT |
| 2 | 209319120 | 24286121 | 7592555 | A | G | CTCTCTCTCTCTTTRGGATTCTAAGGATA |
| 3 | 150732427 | 24608473 | 9858354 | G | T | AATAATAGCCTATTKTATACAACCCAACT |
| X | 109184264 | 24727709 | 2791640 | A | T | ATGCATCCTCTTGGWTAAGGATTCCTGTA |
| 15 | 48059752 | 24006725 | 8039142 | T | C | TAATTACCTTCTTTYCTTATTCAGAGTCC |
| 7 | 19751849 | 23705630 | 6461470 | A | C | ATTTTTTCAAGGGCMAAGATTATTACATA |
| 4 | 150234955 | 23963385 | 17025527 | A | G | CTGTCTTCACTGATRCCATGTTGTTTGAG |
| 15 | 70932755 | 24049569 | 4777560 | A | C | CTTCCAATCAAACAMCCTCCAATCATTCT |
| 18 | 59164885 | 24492133 | 2849372 | G | A | TCGCTTCCTAGATTRGTATTCTCGCTATG |
| 7 | 19336733 | 23704764 | 6963439 | A | G | TTACCAGCCTATCCRTTTTCTGACAAGTT |
| X | 79094700 | 24729919 | 195289 | G | A | AGTCCCCTGCTTCTRAGTAAGTGACTCAT |
| 3 | 150749369 | 24608477 | 9836653 | T | C | TCCATTTAAGTGAAYGGGTAAGGCCTCCC |
| 2 | 29343135 | 23225771 | 12466995 | G | A | TGCCCCGTGGTAACRTGATGGCCTCAGCA |
| X | 79145439 | 24729920 | 1008201 | A | G | CTTGATAGTAGGCTRTACAACTGTTCAAT |
| 8 | 60745793 | 23987913 | 10957105 | T | G | ATACTTTTTAAAAAKGATGACATGATAAA |
| 2 | 209314072 | 14888883 | 10497904 | T | C | CAGTTTTCTAGTCTYGATATTTTTCTTTA |
| 15 | 45925000 | 23995846 | 671291 | G | A | GTGAGAAAAAAGAARTTGACTGAGCAAAT |
| 7 | 19389806 | 23704936 | 4337996 | T | A | CACTACAAACATATWCACCAATTATAAAA |
| 15 | 46939299 | 23433954 | 7169897 | G | C | CTTGCTAGTCAGGCSTCATATCCGGAGAC |
| 19 | 37719854 | 24691720 | 10500261 | A | G | TTCGCCAAAAGTAARATACTATTACCAGA |
| 4 | 150230450 | 23963359 | 17025520 | A | G | ACTCCTTCCACTACRTGATACCTTCAGCT |
| 11 | 88154001 | 23970884 | 10734172 | T | C | TGTGCAGAGGCTTAYTTTGAAGAGCATGT |
| 6 | 90356598 | 23458139 | 6933010 | G | C | TACTCTGAAGATCTSGGAAGCTGTAGGTT |
| 12 | 22099211 | 23425783 | 6416226 | G | A | TGTTACAGATCCAARGGAGTATAAAATGT |
| 2 | 104194770 | 24159875 | 10189155 | G | C | TGATAGAAAGGCAASGATGTTGTGAGGAA |
| 3 | 6463648 | 23804683 | 266415 | C | T | ATGACATCGTACTAYGTTGAAAAGTGGCC |
| 16 | 12368517 | 23975124 | 4781212 | T | G | CACTTTGTGCTGTGKTGTTTGCCACTCTG |
| 11 | 88193955 | 23971323 | 4628675 | G | A | TAAAATGGCCATAGRTAAGAAGATAATTA |
| 3 | 6463056 | 23804681 | 266412 | C | T | CCAGCATGTAAAAAYAGAGACATTTCCAA |
| 11 | 88160044 | 23970984 | 10741523 | C | T | GATAAGCTGAGATCYGACATCCAAGCATC |
| 11 | 88191927 | 23971289 | 11021449 | C | T | ATCACTAACAAGAAYGCTTCCAAAGAGAG |
| 2 | 133659933 | 24298590 | 1370594 | C | G | CATAAAAAACCAAASTAGGAAAAGGGAAA |
| X | 101898585 | 24234568 | 5987637 | C | T | ATATGAAGCAGGTAYGTCAAATCAATGTC |
| 14 | 82064963 | 24110862 | 17116937 | C | T | GGCTTCCTTAGACAYATTGAAATAGTCAT |
| 16 | 4380427 | 24481487 | 917304 | C | G | TGCATGTCTCACAGSTGATAGCAGGGTAC |
| 3 | 6463102 | 23804682 | 266413 | C | T | TTGTATTTTGCAGAYTTGTAGTGAAATTG |
| 11 | 88207543 | 23971498 | 620497 | T | C | AGTGCTCATTTCTTYAGACGTGATTTGCA |
| 11 | 88207722 | 23971505 | 495066 | A | G | TTCGATTTTAGGGTRTGAGAATCCTGCCT |
| 13 | 45063288 | 24424984 | 3014933 | A | C | GATAAATTATGCCAMCAATTCTGATAATA |
| 4 | 55196448 | 23365174 | 6837641 | A | G | AGTTTGATCAGAGARAGCTGCCCAGAGGG |
| 3 | 6491544 | 23804698 | 154961 | T | C | ATTTCTGAATCTCAYTGGCATTTTTCTAA |
| 4 | 89895547 | 23887077 | 2915428 | C | T | AATTGAGAACCTTCYCTGAGGACAAGTCA |
| 15 | 50659822 | 23742984 | 2414160 | C | G | AAAGTGCTCAGAAASGTTGGAAGACTGTT |
| 12 | 75951982 | 23968396 | 10506725 | C | T | CAAATCAAAAGATAYTCAGTTTGCCACTG |
| 9 | 30862374 | 24695557 | 7863381 | G | T | GGTCATTTGTCCTTKTTTGCTCCACMCC |
| 15 | 46950245 | 24001371 | 10519193 | T | C | TGAAGACAAGTAATYATTGAAGTGTTTTT |
| 15 | 45783028 | | 11634811 | G | A | AAGGAACCCACTACRTAGCAACCCAATTT |
| 9 | 1344628 | 23374301 | 1360510 | A | G | TTTCTACAAGGACARTTCTTTGCCTTTAG |
| 1 | 86696528 | | 1321685 | A | G | TTTTATGACTGTGCRTCCATCAAATTTAC |
| X | 79148533 | 24233649 | 1005295 | C | T | TATAATTTCCTCAAYGTCAAAACTAACAG |
| X | 95453891 | 24233829 | 17333535 | G | A | GAACACCGTCTTGGRTGTCAAAAAGACTT |
| X | 95532010 | 24233851 | 7055508 | G | A | TAATACTAGACAACRTGGTAATGATAGGA |
| X | 26730535 | 24235030 | 5926783 | G | A | TATTTGACATCTCTRTAATTTTCCTATCT |
| 3 | 86466914 | 23913456 | 9848250 | T | G | TATTTTTTACATTAKAAATCTCCTGAATT |
| 3 | 86398988 | 23285970 | 6790827 | A | G | AGCCACTAATAATTRCTTTTTCAGTGAAT |
| 15 | 46213776 | | 1426654 | G | A | AGGATGTTGCAGGCRCAACTTTCATGGCA |
| 15 | 46282800 | 23997829 | 1320052 | C | T | GATTTAGAACATATYTGTTATTAGCTATG |
| 15 | 46800835 | 24443789 | 12914304 | T | A | ATGACAGTGAGTTTWGCCAGCTGGAACCA |
| 15 | 46778158 | | 7174374 | A | G | CTGATTAACAAACCRTTAGTAATTCCCTT |
| 15 | 46861831 | 24000792 | 2289179 | T | C | AAAGTTCTGCTTTAYTACTACTGTCTCTT |
| 15 | 46890536 | 24000995 | 2304546 | C | T | TACCCTGGCTCTAGYCCACTAGCTCCTTC |
| 15 | 45702304 | 24435811 | 1435752 | C | T | GGGGTTGGTATTGAYAAGGCCACCTGAGG |
| 15 | 47984948 | 24006429 | 8023809 | A | G | TAGGAGTATAGAGARCAACTTTGAGCAAT |
| 15 | 47025722 | 24445467 | 12898878 | C | T | ACAGAGTTTCTGCTYTTTCACTTGCTTAG |
| X | 8416689 | 23820130 | 16985079 | C | G | AGAAGTATAGCAGGSTTTATGTAGACCAG |
| 15 | 46487221 | 23429859 | 2114438 | T | C | TTATTAGCAGTTAGYTGAAACAACAGATT |
| 15 | 25852901 | 23752539 | 977588 | C | A | TCTTACTACAACAGMAACATTTTAAAAAG |
| 15 | 47001348 | 23434395 | 7164451 | A | G | CAGGAAATTGCTCARTATGGGAGACTTAG |
| 15 | 48043671 | 24006583 | 11070739 | C | G | CTGGTCTGAATCTGSAATGCTGTATGGCT |
| X | 38037118 | 23823774 | 991916 | A | G | TGTGCCCAAACTCCRAAGTTTTTTCCAAT |
| 15 | 45529635 | 23992553 | 1496917 | G | T | ACCCTCCCAAGGTGKGCTCACATTAAATG |
| 15 | 45661803 | 24435261 | 10519132 | G | A | TGACAGTGGATTACRAGGCCACACCATGA |
| X | 38030936 | 24230180 | 17274141 | C | T | AGATGTTCTAATACYTGTCTTCCTCCAGA |
| 17 | 17947264 | 23640492 | 854809 | T | C | GTCAGGACACAGCTYGGGGTCACGGCGCA |
| 15 | 48013605 | | 2452524 | G | T | GGATTCCATCAGATKGTGGTCAAAGAACT |
| X | 38070062 | 23823796 | 5917598 | T | C | TTATTGTTACTCACYTCCATTGCTACTAG |

TABLE 2

| Genomic | | | | | | | |
|---|---|---|---|---|---|---|---|
| Location (NCBI | | Location within gene | | Proximate gene I | | Proximate gene II | |
| Build 35) | | | Location in gene | | Distance | | Distance |
| Chr | Position | Gene | transcript | Gene | (kb) | Gene | (kb) |
| 15 | 46179457 | | | SEMA6D | 326 | SLC24A5 | 21 |
| 15 | 46275146 | LOC400369 | intron | MYEF2 | 17 | SLC12A1 | 12 |
| 15 | 46258816 | | | MYEF2 | 1 | LOC400369 | 12 |
| 15 | 46056053 | | | SEMA6D | 202 | SLC24A5 | 144 |
| 15 | 46420445 | DUT | intron | SLC12A1 | 38 | FBN1 | 69 |
| 15 | 46098702 | | | SEMA6D | 245 | SLC24A5 | 102 |
| 15 | 46087470 | | | SEMA6D | 234 | SLC24A5 | 113 |
| 15 | 46473467 | | | DUT | 51 | FBN1 | 16 |
| 15 | 46049012 | | | SEMA6D | 195 | SLC24A5 | 151 |
| 15 | 46097633 | | | SEMA6D | 244 | SLC24A5 | 103 |
| 15 | 46051787 | | | SEMA6D | 198 | SLC24A5 | 149 |
| 15 | 46472393 | | | DUT | 50 | FBN1 | 17 |
| 15 | 46306954 | SLC12A1 | intron | LOC400369 | 24 | DUT | 105 |
| 15 | 46157395 | | | SEMA6D | 304 | SLC24A5 | 43 |
| 15 | 46971973 | RaLP | intron | CRI1 | 12 | KIAA0256 | 96 |
| 5 | 33987450 | MATP | exon, non- | SALPR | 13 | AMACR | 36 |
| | | | synonymous | | | | |
| 15 | 46986684 | RaLP | intron | CRI1 | 27 | KIAA0256 | 81 |
| 15 | 46313654 | SLC12A1 | intron | LOC400369 | 30 | DUT | 98 |
| 15 | 45957669 | | | SEMA6D | 104 | SLC24A5 | 243 |
| 15 | 46861195 | KIAA0912 | intron | FBN1 | 137 | RaLP | 42 |
| 15 | 46843962 | KIAA0912 | intron | FBN1 | 120 | RaLP | 59 |
| 15 | 46827089 | KIAA0912 | intron | FBN1 | 103 | RaLP | 76 |
| 11 | 88551344 | TYR | exon, non- | GRM5 | 131 | NOX4 | 148 |
| | | | synonymous | | | | |
| 15 | 46521916 | FBN1 | intron | DUT | 99 | KIAA0912 | 296 |
| 15 | 46039330 | | | SEMA6D | 186 | SLC24A5 | 161 |
| 15 | 47018806 | RaLP | intron | CRI1 | 59 | KIAA0256 | 49 |
| 15 | 46055778 | | | SEMA6D | 202 | SLC24A5 | 145 |
| 15 | 46157464 | | | SEMA6D | 304 | SLC24A5 | 43 |
| 15 | 46930524 | RaLP | intron | KIAA0912 | 40 | CRI1 | 27 |
| 15 | 47009173 | RaLP | intron | CRI1 | 50 | KIAA0256 | 59 |
| 15 | 46979618 | RaLP | intron | CRI1 | 20 | KIAA0256 | 89 |
| 15 | 46637949 | FBN1 | intron | DUT | 215 | KIAA0912 | 179 |
| 15 | 46569855 | FBN1 | intron | DUT | 147 | KIAA0912 | 248 |
| 15 | 46963495 | RaLP | intron | CRI1 | 4 | KIAA0256 | 105 |
| 15 | 46731907 | | | FBN1 | 8 | KIAA0912 | 86 |
| 15 | 45903689 | | | SEMA6D | 50 | SLC24A5 | 297 |
| 15 | 45922856 | | | SEMA6D | 69 | SLC24A5 | 278 |
| 15 | 46791064 | | | FBN1 | 67 | KIAA0912 | 26 |
| 2 | 126436772 | | | CNTNAP5 | 1048 | GYPC | 693 |
| 15 | 45965538 | | | SEMA6D | 112 | SLC24A5 | 235 |

| | | Location within gene | | | | | |
|---|---|---|---|---|---|---|---|
| Genomic | | transcript | | | | | |
| Location (NCBI | | | Location | Proximate gene I | | Proximate gene II | |
| Build 35) | | | in gene | | Distance | | Distance |
| Chr | Position | Gene | transcript | Gene | (kb) | Gene | (kb) |
| 2 | 126436342 | | | CNTNAP5 | 1047 | GYPC | 694 |
| 15 | 46892226 | | | KIAA0912 | 2 | RaLP | 11 |
| 15 | 46089356 | | | SEMA6D | 236 | SLC24A5 | 111 |
| 15 | 45970045 | | | SEMA6D | 116 | SLC24A5 | 230 |
| 15 | 46800217 | | | FBN1 | 76 | KIAA0912 | 17 |
| 2 | 126433598 | | | CNTNAP5 | 1044 | GYPC | 696 |
| 18 | 36327583 | | | LOC388474 | 599 | PIK3C3 | 1462 |
| 15 | 46918903 | RaLP | intron | KIAA0912 | 28 | CRI1 | 39 |
| 7 | 19893304 | | | MGC42090 | 351 | 7A5 | 60 |
| 15 | 44862136 | | | LOC145660 | 852 | SEMA6D | 936 |
| 14 | 23885591 | | | RIPK3 | 7 | NFATC4 | 22 |
| 15 | 25671898 | | | LOC390550 | 292 | OCA2 | 2 |
| X | 109181255 | FLJ22679 | intron | ACSL4 | 398 | AMMECR1 | 67 |
| 11 | 88159190 | GRM5 | intron | CTSC | 449 | TYR | 391 |
| 11 | 88154670 | GRM5 | intron | CTSC | 444 | TYR | 396 |
| 15 | 46034246 | | | SEMA6D | 181 | SLC24A5 | 166 |
| 15 | 46834784 | KIAA0912 | intron | FBN1 | 110 | RaLP | 68 |
| 5 | 172955019 | | | LOC389345 | 111 | FAM44B | 12 |
| 14 | 82033421 | | | SEL1L | 964 | LOC283583 | 3031 |
| 15 | 45944278 | | | SEMA6D | 91 | SLC24A5 | 256 |
| 8 | 17415668 | | | MTMR7 | 82 | SLC7A2 | 25 |
| 15 | 46837115 | KIAA0912 | intron | FBN1 | 113 | RaLP | 66 |
| 14 | 82039583 | | | SEL1L | 970 | LOC283583 | 3025 |
| 11 | 88208027 | GRM5 | intron | CTSC | 497 | TYR | 342 |
| 16 | 4154994 | | | ADCY9 | 50 | SRL | 27 |
| 15 | 44942842 | | | LOC145660 | 932 | SEMA6D | 855 |
| X | 118758045 | UPF3B | intron | LOC158796 | 19 | ZNF183 | 28 |
| 15 | 46974966 | RaLP | intron | CRI1 | 15 | KIAA0256 | 93 |
| 3 | 125017560 | MYLK | intron | MYLK | 115 | FLJ12892 | 98 |
| X | 118748996 | | | LOC158796 | 10 | UPF3B | 1 |
| 11 | 88193818 | GRM5 | intron | CTSC | 483 | TYR | 356 |
| 15 | 44939404 | | | LOC145660 | 929 | SEMA6D | 859 |
| 15 | 58988635 | RORA | intron | RORA | 282 | LOC440283 | 39 |
| X | 33404293 | | | DMD | 287 | LOC158724 | 503 |
| 2 | 76405736 | | | C2orf3 | 556 | LRRTM4 | 1250 |
| 2 | 209313998 | | | PTHR2 | 129 | LOC130195 | 449 |
| 6 | 48642656 | | | LOC389395 | 417 | MUT | 864 |
| 18 | 59141498 | | | BCL2 | 4 | FVT1 | 7 |
| 2 | 209319120 | | | PTHR2 | 134 | LOC130195 | 444 |
| 3 | 150732427 | TAZ | intron | TM4SF4 | 29 | LOC440983 | 170 |
| X | 109184264 | FLJ22679 | intron | ACSL4 | 401 | AMMECR1 | 63 |
| 15 | 48059752 | ATP8B4 | intron | MDS009 | 337 | SLC27A2 | 202 |
| 7 | 19751849 | | | MGC42090 | 210 | 7A5 | 202 |
| 4 | 150234955 | | | NR3C2 | 514 | LOC285423 | 474 |
| 15 | 70932755 | | | ADP-GK | 70 | NEO1 | 199 |
| 18 | 59164885 | FVT1 | intron | BCL2 | 28 | VPS4B | 43 |
| 7 | 19336733 | | | FERD3L | 378 | TWISTNB | 172 |
| X | 79094700 | | | TBX22 | 1 | MGC26999 | 387 |
| 3 | 150749369 | TAZ | intron | TM4SF4 | 46 | LOC440983 | 153 |
| 2 | 29343135 | ALK | intron | FLJ21069 | 25 | YPEL5 | 938 |
| X | 79145439 | | | TBX22 | 52 | MGC26999 | 337 |
| 8 | 60745793 | | | TOX | 551 | CAB | 518 |
| 2 | 209314072 | | | PTHR2 | 129 | LOC130195 | 449 |
| 15 | 45925000 | | | SEMA6D | 71 | SLC24A5 | 275 |
| 7 | 19389806 | | | FERD3L | 432 | TWISTNB | 119 |
| 15 | 46939299 | RaLP | intron | KIAA0912 | 49 | CRI1 | 18 |
| 19 | 37719854 | | | LOC147991 | 53 | POCD5 | 44 |
| 4 | 150230450 | | | NR3C2 | 509 | LOC285423 | 478 |
| 11 | 88154001 | GRM5 | intron | CTSC | 443 | TYR | 396 |
| 6 | 90356598 | ANKRD6 | intron | RRAGD | 178 | DJ12208.2 | 47 |
| 12 | 22099211 | CMAS | intron | ABCC9 | 118 | SIAT8A | 146 |
| 2 | 104194770 | | | FLJ30294 | 1302 | LOC150568 | 315 |
| 3 | 6463648 | | | EDEM1 | 1227 | GRM7 | 414 |
| 16 | 12368517 | LOC92017 | intron | FLJ12363 | 314 | FLJ11151 | 296 |
| 11 | 88193955 | GRM5 | intron | CTSC | 483 | TYR | 356 |
| 3 | 6463056 | | | EDEM1 | 1226 | GRM7 | 415 |
| 11 | 88160044 | GRM5 | intron | CTSC | 449 | TYR | 390 |
| 11 | 88191927 | GRM5 | intron | CTSC | 481 | TYR | 358 |
| 2 | 133659933 | FLJ34870 | intron | NAP5 | 287 | MGAT5 | 1183 |
| X | 101898585 | | | KIAA1701 | 85 | LOC286526 | 100 |
| 14 | 82064963 | | | SEL1L | 995 | LOC283583 | 3000 |
| 16 | 4380427 | FLJ22021 | intron | LOC114990 | 6 | DNAJA3 | 35 |
| 3 | 6463102 | | | EDEM1 | 1226 | GRM7 | 415 |
| 11 | 88207543 | GRM5 | intron | CTSC | 497 | TYR | 343 |
| 11 | 88207722 | GRM5 | intron | CTSC | 497 | TYR | 343 |
| 13 | 45063288 | FLJ32682 | intron | COG3 | 55 | NURIT | 111 |
| 4 | 55196448 | | | LOC254938 | 43 | KIT | 169 |
| 3 | 6491544 | | | EDEM1 | 1255 | GRM7 | 386 |
| 4 | 89895547 | | | MGC14156 | 93 | NAP1L5 | 79 |
| 15 | 50659822 | | | ARPP-19 | 11 | FLJ10980 | 1 |
| 12 | 75951982 | E2F7 | intron | CSRP2 | 177 | NAV3 | 776 |
| 9 | 30862374 | | | LOC441392 | 40 | LOC138412 | 382 |
| 15 | 46950245 | RaLP | intron | KIAA0912 | 60 | CRI1 | 7 |
| 15 | 45783028 | | | LO145660 | 1772 | SEMA6D | 15 |
| 9 | 1344628 | | | DMRT2 | 297 | SMARCA2 | 661 |
| 1 | 86696528 | | | CLCA1 | 19 | CLCA4 | 28 |
| X | 79148533 | | | TBX22 | 55 | MGC26999 | 334 |
| X | 95453891 | | | LOC401606 | 418 | DIAPH2 | 292 |
| X | 95532010 | | | LOC401606 | 497 | DIAPH2 | 214 |
| X | 26730535 | | | MAGEB5 | 734 | FLJ32867 | 507 |
| 3 | 86466914 | | | IGSF4D | 267 | FLJ38507 | 603 |
| 3 | 86398988 | | | IGSF4D | 199 | FLJ38507 | 671 |
| 15 | 46213776 | SLC24A5 | exon, non- | SEMA6D | 360 | MYEF2 | 6 |
| | | synonymous | | | | | |
| 15 | 46282800 | LOC400369 | 3′UTR | MYEF2 | 25 | SLC12A1 | 4 |
| 15 | 46800835 | | | FBN1 | 76 | KIAA0912 | 17 |
| 15 | 46778158 | | | FBN1 | 54 | KIAA0912 | 39 |
| 15 | 46861831 | KIAA0912 | intron | FBN1 | 137 | RaLP | 41 |
| 15 | 46890536 | KIAA0912 | 5′UTR | FBN1 | 166 | RaLP | 13 |
| 15 | 45702304 | | | LOC145660 | 1692 | SEMA6D | 96 |
| 15 | 47984948 | ATP8B4 | intron | MDS009 | 262 | SLC27A2 | 277 |
| 15 | 47025722 | RaLP | intron | CRI1 | 66 | KIAA0256 | 42 |
| X | 8416689 | KAL1 | intron | LOC401578 | 172 | FAM9A | 152 |
| 15 | 46487221 | | | DUT | 64 | FBN1 | 2 |
| 15 | 25852901 | OCA2 | intron | LOC390550 | 473 | HERC2 | 177 |
| 15 | 47001348 | RaLP | intron | CRI1 | 42 | KIAA0256 | 67 |
| 15 | 48043671 | ATP8B4 | intron | MDS009 | 321 | SLC27A2 | 218 |
| X | 38037118 | | | OTC | 0 | LOC392443 | 63 |
| 15 | 45529635 | | | LOC145660 | 1519 | SEMA6D | 268 |
| 15 | 45661803 | | | LOC145660 | 1651 | SEMA6D | 136 |
| X | 38030936 | OTC | intron | RPGR | 88 | LOC392443 | 69 |
| 17 | 17947264 | DRG2 | intron | C17orf39 | 35 | MYO15A | 5 |
| 15 | 48013605 | ATP8B4 | exon, non- | MDS009 | 290 | SLC27A2 | 248 |
| | | | synonymous | | | | |
| X | 38070062 | | | OTC | 33 | LOC392443 | 30 |

TABLE 3

| Gene ID in | |
|---|---|
| NCBI Gene | |
| database | Gene Name |
| 115 | ADCY9 |
| 238 | ALK |
| 596 | BCL2 |
| 767 | CA8 |
| 1075 | CTSC |
| 1179 | CLCA1 |
| 1466 | CSRP2 |
| 1730 | DIAPH2 |
| 1756 | DMD |
| 1819 | DRG2 |
| 1854 | DUT |
| 2182 | ACSL4 |
| 2200 | FBN1 |
| 2531 | FVT1 |
| 2915 | GRM5 |
| 2917 | GRM7 |
| 2995 | GYPC |
| 3730 | KAL1 |
| 3815 | KIT |
| 4249 | MGAT5 |
| 4306 | NR3C2 |
| 4594 | MUT |
| 4638 | MYLK |
| 4756 | NEO1 |
| 4776 | NFATC4 |
| 4948 | OCA2 |
| 5009 | OTC |
| 5289 | PIK3C3 |
| 5746 | PTHR2 |
| 6095 | RORA |
| 6103 | RPGR |
| 6345 | SRL |
| 6400 | SEL1L |
| 6489 | SIAT8A/ST8SIA1 |
| 6542 | SLC7A2 |
| 6557 | SLC12A1 |
| 6595 | SMARCA2 |
| 6936 | C2orf3 |
| 7104 | TM4SF4 |
| 7299 | TYR |
| 7737 | ZNF183/RNF113A |
| 8924 | HERC2 |
| 9093 | DNAJA3 |
| 9108 | MTMR7 |
| 9141 | PDCD5 |
| 9525 | VPS4B |
| 9695 | EDEM1 |
| 9728 | KIAA0256 |
| 9760 | TOX |
| 9949 | AMMECR1 |
| 10060 | ABCC9 |
| 10655 | DMRT2 |
| 10776 | ARPP-19 |
| 11001 | SLC27A2 |
| 11035 | RIPK3 |
| 22802 | CLCA4 |
| 22881 | ANKRD6 |
| 22995 | KIAA0912 |
| 23600 | AMACR |
| 23741 | CRI1 |
| 25937 | TAZ/WWTR1 |
| 50507 | NOX4 |
| 50804 | MYEF2 |
| 50945 | TBX22 |
| 51151 | MATP |
| 51168 | MYO15A |
| 51289 | SALPR/RLN3R1/RXFP3 |
| 51646 | YPEL5 |
| 55313 | FLJ11151 |
| 55907 | CMAS |
| 56204 | FLJ10980 |
| 56986 | MDS009/DTWD1 |
| 57226 | DJ122O8.2 |
| 58528 | RRAGD |
| 64770 | FLJ12892/CCDC14 |
| 65109 | UPF3B |
| 79018 | C17orf39 |
| 79585 | FLJ22021/COR07 |
| 79745 | FLJ21069/RSNL2 |
| 79895 | ATP8B4 |
| 80031 | SEMA6D |
| 80059 | LRRTM4 |
| 80823 | KIAA1701/BHLHB9 |
| 83440 | ADP-GK |
| 83548 | COG3 |
| 84127 | FLJ12363/RUNDC2A |
| 84187 | FLJ22679/RP13-360B22.2 |
| 84992 | MGC14156 |
| 89795 | NAV3 |
| 91272 | FAM44B |
| 92017 | LOC92017 |
| 114990 | LOC114990/SLITL2/VASN |
| 129684 | CNTNAP5 |
| 130195 | LOC130195 |
| 130827 | FLJ30294 |
| 138412 | LOC138412 |
| 139420 | FLJ32867 |
| 144455 | E2F7 |
| 145660 | LOC145660 |
| 147991 | LOC147991/DPY19L3 |
| 150568 | LOC150568 |
| 158724 | LOC158724/FAM47A |
| 158796 | LOC158796 |
| 169966 | MGC26999/FAM46D |
| 171482 | FAM9A |
| 220081 | FLJ32682 |
| 220082 | NURIT |
| 221830 | TWISTNB |
| 222894 | FERD3L |
| 253559 | IGSF4D |
| 254938 | LOC254938 |
| 256130 | MGC42090 |
| 266812 | NAP1L5 |
| 283583 | LOC283583 |
| 283652 | SLC24A5 |
| 285423 | LOC285423 |
| 286526 | LOC286526 |
| 344148 | NAP5 |
| 346389 | 7A5 |
| 347541 | MAGEB5 |
| 388474 | LOC388474 |
| 389136 | FLJ38507/VGL-3/VGLL3 |
| 389345 | LOC389345 |
| 389395 | LOC389395 |
| 390550 | LOC390550 |
| 392443 | LOC392443 |
| 399694 | RaLP |
| 400369 | LOC400369/DKFZp781M2440 |
| 401013 | FLJ34870 |
| 401578 | LOC401578 |
| 401606 | LOC401606 |
| 440283 | LOC440283 |
| 440983 | LOC440983 |
| 441392 | LOC441392 |

TABLE 4

| | | | Sample Set 1 | | | | Sample Set 2 | | | | | | |
|---|---|---|---|---|---|---|---|---|---|---|---|---|---|
| | | | | | Singificance of | | | | Singificance of | | Joint Association Analysis | | |
| Genomic Location of SNP | | | Reference | | association | | Reference | | association | | Allele | | |
| (NCBI Build 35) | | | Allele | | | False | Allele | | | False | Freq. | | |
| | | reference | frequency | | | Discovery | frequency | | | Discovery | Diff. | | False |
| Chr | Position | allele | D | L | p-value | Rate | D | L | p-value | Rate | (D − L) | p-value | Discovery Rate |
| 15 | 46179457 | A | 0.47 | 0.92 | 5.51E−27 | 8.27E−21 | 0.51 | 0.87 | 4.34E−13 | 6.09E−10 | −0.43 | 2.06E−30 | 3.34E−24 |
| 15 | 46275146 | C | 0.33 | 0.05 | 6.68E−17 | 5.02E−11 | 0.31 | 0.06 | 7.17E−09 | 3.35E−06 | 0.28 | 3.49E−19 | 2.83E−13 |
| 15 | 46258816 | A | 0.33 | 0.05 | 1.27E−16 | 6.34E−11 | 0.31 | 0.06 | 6.19E−09 | 3.35E−06 | 0.28 | 5.75E−19 | 3.11E−13 |
| 15 | 46056053 | G | 0.55 | 0.25 | 1.60E−13 | 4.80E−08 | 0.51 | 0.25 | 1.59E−06 | 5.57E−04 | 0.29 | 6.53E−15 | 2.65E−09 |
| 15 | 46420445 | C | 0.66 | 0.35 | 5.71E−14 | 2.15E−08 | 0.59 | 0.42 | 1.01E−03 | 4.57E−02 | 0.28 | 1.11E−13 | 3.61E−08 |
| 15 | 46098702 | A | 0.65 | 0.89 | 9.08E−12 | 2.27E−06 | 0.68 | 0.88 | 3.37E−06 | 9.46E−04 | −0.23 | 3.17E−13 | 8.57E−08 |
| 15 | 46087470 | T | 0.52 | 0.78 | 3.60E−11 | 7.73E−06 | 0.52 | 0.77 | 4.87E−06 | 1.01E−03 | −0.25 | 1.49E−12 | 3.44E−07 |
| 15 | 46473467 | A | 0.57 | 0.81 | 3.82E−10 | 7.17E−05 | 0.64 | 0.76 | 9.49E−03 | 2.35E−01 | −0.21 | 9.88E−10 | 2.00E−04 |
| 15 | 46049012 | C | 0.33 | 0.13 | 8.45E−09 | 1.27E−03 | 0.30 | 0.15 | 3.90E−04 | 2.73E−02 | 0.19 | 1.73E−09 | 3.12E−04 |
| 15 | 46097633 | G | 0.80 | 0.95 | 2.47E−08 | 2.86E−03 | 0.80 | 0.94 | 1.36E−04 | 1.12E−02 | −0.15 | 2.58E−09 | 4.19E−04 |
| 15 | 46051787 | T | 0.33 | 0.13 | 1.67E−08 | 2.09E−03 | 0.31 | 0.16 | 4.22E−04 | 2.82E−02 | 0.19 | 3.20E−09 | 4.72E−04 |
| 15 | 46472393 | C | 0.41 | 0.19 | 2.18E−09 | 3.64E−04 | 0.35 | 0.24 | 1.26E−02 | 2.55E−01 | 0.19 | 4.80E−09 | 6.48E−04 |
| 15 | 46306954 | G | 0.84 | 0.97 | 5.86E−08 | 6.29E−03 | 0.85 | 0.97 | 1.08E−04 | 1.04E−02 | −0.13 | 4.93E−09 | 6.15E−04 |
| 15 | 46157395 | G | 0.83 | 0.96 | 1.14E−07 | 1.07E−02 | 0.87 | 0.97 | 9.84E−04 | 4.57E−02 | −0.12 | 2.68E−08 | 3.10E−03 |
| 15 | 46971973 | G | 0.80 | 0.63 | 1.52E−06 | 1.04E−01 | 0.84 | 0.63 | 1.11E−05 | 1.73E−03 | 0.18 | 3.75E−08 | 4.06E−03 |
| 5 | 33987450 | C | 0.97 | 0.83 | 8.11E−08 | 8.12E−03 | 0.94 | 0.85 | 9.15E−03 | 2.35E−01 | 0.12 | 7.28E−08 | 7.38E−03 |
| 15 | 46986684 | G | 0.69 | 0.51 | 5.49E−06 | 2.36E−01 | 0.72 | 0.48 | 5.06E−06 | 1.01E−03 | 0.20 | 8.39E−08 | 8.00E−03 |
| 15 | 46313654 | G | 0.45 | 0.23 | 1.35E−08 | 1.84E−03 | 0.40 | 0.31 | 8.77E−02 | 5.34E−01 | 0.19 | 1.12E−07 | 1.00E−02 |
| 15 | 45957669 | C | 0.59 | 0.40 | 2.21E−06 | 1.33E−01 | 0.58 | 0.38 | 1.28E−04 | 1.12E−02 | 0.19 | 1.46E−07 | 1.24E−02 |
| 15 | 46861195 | A | 0.59 | 0.76 | 3.74E−06 | 1.75E−01 | 0.57 | 0.78 | 6.04E−05 | 7.06E−03 | −0.18 | 1.67E−07 | 1.35E−02 |
| 15 | 46843962 | T | 0.59 | 0.75 | 6.02E−06 | 2.51E−01 | 0.56 | 0.77 | 2.71E−05 | 3.80E−03 | −0.18 | 1.83E−07 | 1.41E−02 |
| 15 | 46827089 | T | 0.59 | 0.76 | 6.08E−06 | 2.47E−01 | 0.56 | 0.76 | 5.02E−05 | 6.39E−03 | −0.18 | 2.41E−07 | 1.77E−02 |
| 11 | 88551344 | C | 0.96 | 0.84 | 5.05E−07 | 4.46E−02 | 0.94 | 0.87 | 3.01E−02 | 3.72E−01 | 0.11 | 7.85E−07 | 5.53E−02 |
| 15 | 46521916 | G | 0.49 | 0.31 | 2.62E−06 | 1.46E−01 | 0.53 | 0.37 | 2.48E−03 | 9.91E−02 | 0.18 | 8.36E−07 | 5.65E−02 |
| 15 | 46039330 | T | 0.22 | 0.08 | 3.02E−06 | 1.51E−01 | 0.19 | 0.09 | 4.57E−03 | 1.73E−01 | 0.13 | 1.07E−06 | 6.94E−02 |
| 15 | 47018806 | G | 0.32 | 0.14 | 5.40E−07 | 4.51E−02 | 0.26 | 0.18 | 6.28E−02 | 4.70E−01 | 0.15 | 1.15E−06 | 7.16E−02 |
| 15 | 46055778 | G | 0.82 | 0.67 | 1.32E−05 | 4.12E−01 | 0.80 | 0.64 | 5.99E−04 | 3.50E−02 | 0.15 | 1.50E−06 | 8.98E−02 |
| 15 | 46157464 | A | 0.89 | 0.98 | 2.25E−06 | 1.30E−01 | 0.90 | 0.97 | 7.89E−03 | 2.35E−01 | −0.09 | 1.51E−06 | 8.72E−02 |
| 15 | 46930524 | G | 0.69 | 0.53 | 1.07E−04 | 1 | 0.75 | 0.51 | 6.57E−06 | 1.15E−03 | 0.18 | 1.82E−06 | 1.02E−01 |
| 15 | 47009173 | G | 0.76 | 0.59 | 4.93E−06 | 2.25E−01 | 0.74 | 0.61 | 6.75E−03 | 2.20E−01 | 0.16 | 2.09E−06 | 1.13E−01 |
| 15 | 46979618 | T | 0.58 | 0.75 | 1.41E−05 | 4.34E−01 | 0.60 | 0.76 | 1.23E−03 | 5.39E−02 | −0.16 | 2.26E−06 | 1.18E−01 |
| 15 | 46637949 | C | 0.47 | 0.30 | 2.25E−05 | 5.92E−01 | 0.53 | 0.34 | 4.48E−04 | 2.86E−02 | 0.17 | 2.31E−06 | 1.17E−01 |
| 15 | 46569855 | T | 0.50 | 0.67 | 1.63E−05 | 4.72E−01 | 0.44 | 0.62 | 8.57E−04 | 4.25E−02 | −0.17 | 2.42E−06 | 1.19E−01 |
| 15 | 46963495 | A | 0.59 | 0.75 | 1.90E−05 | 5.29E−01 | 0.60 | 0.76 | 8.80E−04 | 4.25E−02 | −0.17 | 2.55E−06 | 1.21E−01 |
| 15 | 46731907 | C | 0.39 | 0.24 | 4.57E−05 | 8.58E−01 | 0.54 | 0.35 | 2.10E−04 | 1.56E−02 | 0.17 | 2.74E−06 | 1.24E−01 |
| 15 | 45903689 | T | 0.42 | 0.61 | 3.53E−06 | 1.71E−01 | 0.49 | 0.60 | 2.79E−02 | 3.59E−01 | −0.17 | 3.73E−06 | 1.59E−01 |
| 15 | 45922856 | C | 0.33 | 0.49 | 4.51E−05 | 8.92E−01 | 0.35 | 0.53 | 7.74E−04 | 4.02E−02 | −0.17 | 5.30E−06 | 2.20E−01 |
| 15 | 46791064 | T | 0.56 | 0.41 | 1.60E−04 | 1 | 0.61 | 0.40 | 1.11E−04 | 1.04E−02 | 0.17 | 7.38E−06 | 2.99E−01 |
| 2 | 1.26E+08 | T | 0.61 | 0.43 | 8.40E−06 | 3.15E−01 | 0.58 | 0.48 | 6.55E−02 | 4.80E−01 | 0.16 | 1.33E−05 | 4.89E−01 |
| 15 | 45965538 | T | 0.65 | 0.79 | 8.67E−05 | 1 | 0.67 | 0.82 | 1.73E−03 | 7.36E−02 | −0.14 | 1.39E−05 | 5.00E−01 |
| 2 | 1.26E+08 | A | 0.61 | 0.43 | 9.03E−06 | 3.31E−01 | 0.58 | 0.48 | 6.63E−02 | 4.80E−01 | 0.16 | 1.41E−05 | 4.98E−01 |
| 15 | 46892226 | A | 0.72 | 0.58 | 2.41E−04 | 1 | 0.74 | 0.55 | 2.11E−04 | 1.56E−02 | 0.15 | 1.50E−05 | 5.06E−01 |
| 15 | 46089356 | G | 0.78 | 0.63 | 1.56E−04 | 1 | 0.80 | 0.65 | 4.82E−04 | 2.94E−02 | 0.14 | 1.51E−05 | 5.00E−01 |
| 15 | 45970045 | G | 0.66 | 0.80 | 5.66E−05 | 9.89E−01 | 0.70 | 0.82 | 6.52E−03 | 2.18E−01 | −0.14 | 1.78E−05 | 5.25E−01 |
| 15 | 46800217 | G | 0.22 | 0.10 | 4.36E−05 | 8.74E−01 | 0.21 | 0.11 | 1.12E−02 | 2.45E−01 | 0.11 | 1.93E−05 | 5.49E−01 |
| 2 | 1.26E+08 | A | 0.61 | 0.43 | 1.21E−05 | 3.95E−01 | 0.58 | 0.49 | 7.81E−02 | 5.13E−01 | 0.16 | 2.00E−05 | 5.58E−01 |
| 18 | 36327583 | G | 0.86 | 0.72 | 3.77E−05 | 8.46E−01 | 0.84 | 0.75 | 3.33E−02 | 3.74E−01 | 0.12 | 3.27E−05 | 8.54E−01 |
| 15 | 46918903 | A | 0.71 | 0.58 | 3.81E−04 | 1 | 0.73 | 0.56 | 6.79E−04 | 3.66E−02 | 0.15 | 3.68E−05 | 9.17E−01 |
| 7 | 19893304 | G | 0.18 | 0.32 | 5.66E−05 | 9.78E−01 | 0.19 | 0.28 | 4.22E−02 | 3.96E−01 | −0.13 | 4.91E−05 | 1 |
| 15 | 44862136 | A | 0.65 | 0.50 | 2.08E−04 | 1 | 0.67 | 0.51 | 5.66E−03 | 1.98E−01 | 0.15 | 5.41E−05 | 1 |
| 14 | 23885591 | A | 0.74 | 0.59 | 6.13E−05 | 1 | 0.72 | 0.63 | 6.16E−02 | 4.69E−01 | 0.14 | 6.90E−05 | 1 |
| 15 | 25671898 | T | 0.56 | 0.71 | 9.36E−05 | 1 | 0.58 | 0.68 | 4.26E−02 | 3.96E−01 | −0.14 | 7.89E−05 | 1 |
| X | 1.09E+08 | A | 0.69 | 0.52 | 1.25E−04 | 1 | 0.64 | 0.53 | 3.85E−02 | 3.96E−01 | 0.15 | 8.35E−05 | 1 |
| 11 | 88159190 | C | 0.71 | 0.56 | 1.66E−04 | 1 | 0.68 | 0.57 | 2.67E−02 | 3.49E−01 | 0.14 | 9.72E−05 | 1 |
| 11 | 88154670 | A | 0.72 | 0.56 | 1.22E−04 | 1 | 0.68 | 0.58 | 4.28E−02 | 3.96E−01 | 0.14 | 9.72E−05 | 1 |
| 15 | 46034246 | A | 0.25 | 0.39 | 1.40E−04 | 1 | 0.29 | 0.41 | 3.27E−02 | 3.72E−01 | −0.14 | 9.75E−05 | 1 |
| 15 | 46834784 | A | 0.76 | 0.88 | 2.03E−04 | 1 | 0.78 | 0.87 | 2.44E−02 | 3.41E−01 | −0.11 | 1.09E−04 | 1 |
| 5 | 1.73E+08 | G | 0.39 | 0.54 | 1.94E−04 | 1 | 0.40 | 0.52 | 2.50E−02 | 3.41E−01 | −0.14 | 1.14E−04 | 1 |
| 14 | 82033421 | T | 0.55 | 0.69 | 1.84E−04 | 1 | 0.57 | 0.68 | 3.58E−02 | 3.83E−01 | −0.14 | 1.22E−04 | 1 |
| 15 | 45944278 | G | 0.70 | 0.81 | 1.32E−03 | 1 | 0.71 | 0.86 | 6.73E−04 | 3.66E−02 | −0.12 | 1.26E−04 | 1 |
| 8 | 17415668 | T | 0.63 | 0.75 | 6.75E−04 | 1 | 0.63 | 0.77 | 3.64E−03 | 1.42E−01 | −0.13 | 1.27E−04 | 1 |
| 15 | 46837115 | C | 0.24 | 0.12 | 2.63E−04 | 1 | 0.22 | 0.13 | 2.11E−02 | 3.16E−01 | 0.11 | 1.28E−04 | 1 |
| 14 | 82039583 | G | 0.55 | 0.70 | 1.33E−04 | 1 | 0.59 | 0.68 | 6.19E−02 | 4.69E−01 | −0.14 | 1.31E−04 | 1 |
| 11 | 88208027 | A | 0.72 | 0.57 | 1.58E−04 | 1 | 0.68 | 0.58 | 4.96E−02 | 4.16E−01 | 0.14 | 1.32E−04 | 1 |
| 16 | 4154994 | A | 0.65 | 0.79 | 1.79E−04 | 1 | 0.66 | 0.75 | 3.95E−02 | 3.96E−01 | −0.13 | 1.35E−04 | 1 |
| 15 | 44942842 | C | 0.55 | 0.40 | 3.26E−04 | 1 | 0.50 | 0.38 | 1.74E−02 | 2.87E−01 | 0.14 | 1.37E−04 | 1 |
| X | 1.19E+08 | T | 0.39 | 0.55 | 3.42E−04 | 1 | 0.40 | 0.53 | 2.18E−02 | 3.18E−01 | −0.15 | 1.47E−04 | 1 |
| 15 | 46974966 | G | 0.71 | 0.83 | 4.89E−04 | 1 | 0.73 | 0.84 | 1.03E−02 | 2.35E−01 | −0.12 | 1.51E−04 | 1 |
| 3 | 1.25E+08 | C | 0.69 | 0.54 | 2.53E−04 | 1 | 0.66 | 0.56 | 3.53E−02 | 3.83E−01 | 0.13 | 1.57E−04 | 1 |
| X | 1.19E+08 | C | 0.40 | 0.56 | 2.58E−04 | 1 | 0.42 | 0.54 | 4.03E−02 | 3.96E−01 | −0.15 | 1.59E−04 | 1 |
| 11 | 88193818 | T | 0.72 | 0.58 | 2.31E−04 | 1 | 0.69 | 0.58 | 4.25E−02 | 3.96E−01 | 0.14 | 1.68E−04 | 1 |
| 15 | 44939404 | T | 0.54 | 0.40 | 3.76E−04 | 1 | 0.50 | 0.37 | 2.12E−02 | 3.16E−01 | 0.14 | 1.73E−04 | 1 |
| 15 | 58988635 | T | 0.51 | 0.35 | 2.09E−04 | 1 | 0.50 | 0.40 | 5.54E−02 | 4.34E−01 | 0.14 | 1.79E−04 | 1 |
| X | 33404293 | T | 0.70 | 0.82 | 8.98E−04 | 1 | 0.68 | 0.82 | 5.43E−03 | 1.95E−01 | −0.13 | 1.80E−04 | 1 |
| 2 | 76405736 | T | 0.49 | 0.63 | 2.71E−04 | 1 | 0.50 | 0.60 | 4.19E−02 | 3.96E−01 | −0.14 | 1.88E−04 | 1 |
| 2 | 2.09E+08 | C | 0.82 | 0.69 | 2.29E−04 | 1 | 0.77 | 0.68 | 5.67E−02 | 4.38E−01 | 0.12 | 2.00E−04 | 1 |
| 6 | 48642656 | G | 0.73 | 0.84 | 6.58E−04 | 1 | 0.72 | 0.83 | 1.19E−02 | 2.46E−01 | −0.11 | 2.16E−04 | 1 |
| 18 | 59141498 | G | 0.38 | 0.54 | 1.74E−04 | 1 | 0.43 | 0.52 | 1.03E−01 | 5.50E−01 | −0.14 | 2.25E−04 | 1 |
| 2 | 2.09E+08 | A | 0.81 | 0.68 | 2.90E−04 | 1 | 0.77 | 0.68 | 5.67E−02 | 4.38E−01 | 0.12 | 2.44E−04 | 1 |
| 3 | 1.51E+08 | G | 0.80 | 0.68 | 9.04E−04 | 1 | 0.80 | 0.68 | 8.87E−03 | 2.35E−01 | 0.12 | 2.50E−04 | 1 |
| X | 1.09E+08 | A | 0.67 | 0.52 | 2.95E−04 | 1 | 0.61 | 0.52 | 7.35E−02 | 5.01E−01 | 0.14 | 2.53E−04 | 1 |
| 15 | 48059752 | C | 0.50 | 0.62 | 1.75E−03 | 1 | 0.47 | 0.63 | 2.06E−03 | 8.48E−02 | −0.13 | 2.54E−04 | 1 |
| 7 | 19751849 | C | 0.35 | 0.50 | 4.23E−04 | 1 | 0.35 | 0.46 | 4.26E−02 | 3.96E−01 | −0.14 | 2.83E−04 | 1 |
| 4 | 1.5E+08 | G | 0.61 | 0.73 | 9.02E−04 | 1 | 0.61 | 0.74 | 1.42E−02 | 2.63E−01 | −0.12 | 3.09E−04 | 1 |
| 15 | 70932755 | C | 0.70 | 0.82 | 7.24E−04 | 1 | 0.69 | 0.79 | 2.20E−02 | 3.18E−01 | −0.12 | 3.18E−04 | 1 |
| 18 | 59164885 | A | 0.38 | 0.52 | 3.44E−04 | 1 | 0.43 | 0.52 | 7.36E−02 | 5.01E−01 | −0.13 | 3.22E−04 | 1 |
| 7 | 19336733 | A | 0.69 | 0.55 | 3.35E−04 | 1 | 0.70 | 0.60 | 7.19E−02 | 4.97E−01 | 0.13 | 3.28E−04 | 1 |
| X | 79094700 | A | 0.19 | 0.32 | 7.91E−04 | 1 | 0.18 | 0.28 | 2.99E−02 | 3.72E−01 | −0.12 | 3.60E−04 | 1 |
| 3 | 1.51E+08 | T | 0.69 | 0.56 | 6.45E−04 | 1 | 0.69 | 0.59 | 3.61E−02 | 3.83E−01 | 0.13 | 3.68E−04 | 1 |
| 2 | 29343135 | A | 0.47 | 0.61 | 6.45E−04 | 1 | 0.47 | 0.58 | 4.13E−02 | 3.96E−01 | −0.13 | 4.00E−04 | 1 |
| X | 79145439 | G | 0.18 | 0.31 | 8.84E−04 | 1 | 0.19 | 0.29 | 3.15E−02 | 3.72E−01 | −0.12 | 4.12E−04 | 1 |
| 8 | 60745793 | G | 0.44 | 0.58 | 5.88E−04 | 1 | 0.45 | 0.54 | 6.25E−02 | 4.70E−01 | −0.13 | 4.48E−04 | 1 |
| 2 | 2.09E+08 | T | 0.82 | 0.70 | 5.67E−04 | 1 | 0.77 | 0.68 | 5.13E−02 | 4.18E−01 | 0.11 | 4.53E−04 | 1 |
| 15 | 45925000 | A | 0.68 | 0.79 | 8.25E−04 | 1 | 0.68 | 0.78 | 4.35E−02 | 3.96E−01 | −0.11 | 5.06E−04 | 1 |
| 7 | 19389806 | A | 0.37 | 0.51 | 8.55E−04 | 1 | 0.34 | 0.45 | 4.32E−02 | 3.96E−01 | −0.13 | 5.22E−04 | 1 |
| 15 | 46939299 | C | 0.70 | 0.81 | 1.61E−03 | 1 | 0.71 | 0.82 | 1.44E−02 | 2.63E−01 | −0.11 | 5.23E−04 | 1 |
| 19 | 37719854 | A | 0.64 | 0.52 | 1.63E−03 | 1 | 0.64 | 0.52 | 1.41E−02 | 2.63E−01 | 0.12 | 5.29E−04 | 1 |
| 4 | 1.5E+08 | G | 0.62 | 0.73 | 2.70E−03 | 1 | 0.60 | 0.73 | 8.13E−03 | 2.35E−01 | −0.11 | 6.55E−04 | 1 |
| 11 | 88154001 | T | 0.63 | 0.50 | 1.32E−03 | 1 | 0.59 | 0.48 | 3.39E−02 | 3.77E−01 | 0.12 | 6.64E−04 | 1 |
| 6 | 90356598 | G | 0.59 | 0.46 | 8.67E−04 | 1 | 0.62 | 0.53 | 6.55E−02 | 4.80E−01 | 0.12 | 6.78E−04 | 1 |
| 12 | 22099211 | A | 0.46 | 0.59 | 1.62E−03 | 1 | 0.47 | 0.58 | 2.50E−02 | 3.41E−01 | −0.13 | 6.79E−04 | 1 |
| 2 | 1.04E+08 | C | 0.70 | 0.82 | 1.16E−03 | 1 | 0.74 | 0.83 | 4.13E−02 | 3.96E−01 | −0.11 | 7.08E−04 | 1 |
| 3 | 6463648 | T | 0.55 | 0.68 | 1.27E−03 | 1 | 0.57 | 0.67 | 4.88E−02 | 4.15E−01 | −0.12 | 7.62E−04 | 1 |
| 16 | 12368517 | T | 0.52 | 0.39 | 1.23E−03 | 1 | 0.46 | 0.36 | 5.55E−02 | 4.34E−01 | 0.12 | 8.09E−04 | 1 |
| 11 | 88193955 | G | 0.61 | 0.48 | 1.36E−03 | 1 | 0.57 | 0.46 | 5.66E−02 | 4.38E−01 | 0.12 | 8.83E−04 | 1 |
| 3 | 6463056 | T | 0.59 | 0.71 | 2.18E−03 | 1 | 0.58 | 0.69 | 2.65E−02 | 3.49E−01 | −0.12 | 9.16E−04 | 1 |
| 11 | 88160044 | C | 0.66 | 0.53 | 1.73E−03 | 1 | 0.64 | 0.53 | 4.07E−02 | 3.96E−01 | 0.12 | 9.26E−04 | 1 |
| 11 | 88191927 | C | 0.60 | 0.48 | 1.69E−03 | 1 | 0.58 | 0.47 | 4.64E−02 | 3.99E−01 | 0.12 | 9.77E−04 | 1 |
| 2 | 1.34E+08 | G | 0.24 | 0.35 | 1.67E−03 | 1 | 0.20 | 0.30 | 5.04E−02 | 4.16E−01 | −0.11 | 1.04E−03 | 1 |
| X | 1.02E+08 | C | 0.47 | 0.34 | 3.53E−03 | 1 | 0.53 | 0.40 | 1.52E−02 | 2.65E−01 | 0.13 | 1.05E−03 | 1 |
| 14 | 82064963 | T | 0.53 | 0.66 | 1.34E−03 | 1 | 0.56 | 0.66 | 7.14E−02 | 4.97E−01 | −0.12 | 1.05E−03 | 1 |
| 16 | 4380427 | C | 0.43 | 0.32 | 3.91E−03 | 1 | 0.44 | 0.31 | 1.32E−02 | 2.60E−01 | 0.12 | 1.13E−03 | 1 |
| 3 | 6463102 | T | 0.59 | 0.71 | 2.49E−03 | 1 | 0.59 | 0.69 | 3.25E−02 | 3.72E−01 | −0.11 | 1.14E−03 | 1 |
| 11 | 88207543 | T | 0.60 | 0.47 | 1.36E−03 | 1 | 0.56 | 0.47 | 8.91E−02 | 5.38E−01 | 0.12 | 1.16E−03 | 1 |
| 11 | 88207722 | A | 0.60 | 0.47 | 1.67E−03 | 1 | 0.56 | 0.47 | 6.51E−02 | 4.80E−01 | 0.12 | 1.17E−03 | 1 |
| 13 | 45063288 | A | 0.78 | 0.67 | 3.35E−03 | 1 | 0.80 | 0.69 | 2.06E−02 | 3.13E−01 | 0.11 | 1.22E−03 | 1 |
| 4 | 55196448 | G | 0.62 | 0.73 | 4.48E−03 | 1 | 0.63 | 0.75 | 1.18E−02 | 2.46E−01 | −0.11 | 1.25E−03 | 1 |
| 3 | 6491544 | T | 0.39 | 0.27 | 2.46E−03 | 1 | 0.39 | 0.30 | 4.61E−02 | 3.99E−01 | 0.11 | 1.34E−03 | 1 |
| 4 | 89895547 | C | 0.62 | 0.50 | 3.90E−03 | 1 | 0.63 | 0.51 | 2.04E−02 | 3.13E−01 | 0.12 | 1.38E−03 | 1 |
| 15 | 50659822 | G | 0.65 | 0.77 | 3.83E−03 | 1 | 0.62 | 0.73 | 2.94E−02 | 3.72E−01 | −0.12 | 1.38E−03 | 1 |
| 12 | 75951982 | T | 0.70 | 0.80 | 2.64E−03 | 1 | 0.70 | 0.79 | 5.48E−02 | 4.34E−01 | −0.10 | 1.57E−03 | 1 |
| 9 | 30862374 | T | 0.56 | 0.68 | 2.49E−03 | 1 | 0.61 | 0.70 | 7.71E−02 | 5.13E−01 | −0.11 | 1.91E−03 | 1 |
| 15 | 46950245 | T | 0.42 | 0.31 | 5.43E−03 | 1 | 0.40 | 0.30 | 4.09E−02 | 8.79E−02 | 0.11 | 2.51E−03 | 1 |
| 15 | 45783028 | G | | | | | 0.74 | 0.61 | 3.42E−02 | | | | |
| 9 | 1344628 | A | 0.49 | 0.37 | 3.85E−03 | 1 | 0.53 | 0.44 | 8.68E−02 | 5.34E−01 | 0.11 | 2.80E−03 | 1 |
| 1 | 86696528 | G | 0.53 | 0.64 | 6.36E−03 | 1 | 0.53 | 0.63 | 4.12E−02 | 3.96E−01 | −0.11 | 2.94E−03 | 1 |
| X | 79148533 | T | 0.54 | 0.64 | 1.42E−02 | 1 | 0.48 | 0.60 | 2.97E−02 | 1.71E−01 | −0.11 | 5.26E−03 | 1 |
| X | 95453891 | A | 0.61 | 0.76 | 1.67E−04 | 1 | 0.76 | 0.65 | 3.08E−02 | 3.72E−01 | −0.09 | 1.39E−02 | 1 |
| X | 95532010 | A | 0.62 | 0.76 | 5.59E−04 | 1 | 0.77 | 0.64 | 1.64E−02 | 2.77E−01 | −0.08 | 3.42E−02 | 1 |
| X | 26730535 | A | 0.34 | 0.49 | 1.11E−03 | 1 | 0.56 | 0.42 | 1.48E−02 | 2.63E−01 | −0.08 | 6.56E−02 | 1 |
| 3 | 86466914 | T | 0.77 | 0.67 | 6.51E−03 | 1 | 0.67 | 0.76 | 5.09E−02 | 4.17E−01 | 0.05 | 1.14E−01 | 1 |
| 3 | 86398988 | A | 0.74 | 0.63 | 5.16E−03 | 1 | 0.60 | 0.75 | 4.97E−03 | 1.83E−01 | 0.05 | 1.71E−01 | 1 |
| 15 | 46213776 | A | | | | | 0.51 | 0.90 | 4.46E−15 | 2.31E−13 | | | |
| 15 | 46282800 | C | | | | | 0.31 | 0.06 | 7.17E−09 | 1.86E−07 | | | |
| 15 | 46800835 | T | | | | | 0.66 | 0.46 | 3.26E−04 | 5.94E−02 | | | |
| 15 | 46778158 | A | | | | | 0.34 | 0.19 | 7.65E−04 | 6.96E−02 | | | |
| 15 | 46861831 | C | | | | | 0.78 | 0.90 | 1.60E−03 | 9.72E−02 | | | |
| 15 | 46890536 | T | | | | | 0.70 | 0.84 | 1.78E−03 | 3.07E−02 | | | |
| 15 | 45702304 | T | | | | | 0.66 | 0.80 | 3.04E−03 | 1.38E−01 | | | |
| 15 | 47984948 | A | | | | | 0.38 | 0.24 | 3.88E−03 | 1.41E−01 | | | |
| 15 | 47025722 | C | | | | | 0.61 | 0.47 | 5.17E−03 | 1.57E−01 | | | |
| X | 8416689 | C | | | | | 0.85 | 0.73 | 6.08E−03 | 1.71E−01 | | | |
| 15 | 46487221 | T | | | | | 0.48 | 0.34 | 6.57E−03 | 1.71E−01 | | | |
| 15 | 25852901 | C | | | | | 0.82 | 0.69 | 8.25E−03 | 4.34E−01 | | | |
| 15 | 47001348 | G | | | | | 0.24 | 0.36 | 1.30E−02 | 2.36E−01 | | | |
| 15 | 48043671 | G | | | | | 0.61 | 0.73 | 1.42E−02 | 2.36E−01 | | | |
| X | 38037118 | A | | | | | 0.82 | 0.71 | 1.61E−02 | 1.71E−01 | | | |
| 15 | 45529635 | G | | | | | 0.79 | 0.69 | 1.82E−02 | 2.36E−01 | | | |
| 15 | 45661803 | G | | | | | 0.78 | 0.66 | 1.93E−02 | 2.36E−01 | | | |
| X | 38030936 | T | | | | | 0.66 | 0.77 | 2.16E−02 | 1.71E−01 | | | |
| 17 | 17947264 | T | | | | | 0.75 | 0.65 | 3.78E−02 | 9.77E−01 | | | |
| 15 | 48013605 | G | | | | | 0.47 | 0.36 | 4.01E−02 | 2.24E−01 | | | |
| X | 38070062 | T | | | | | 0.70 | 0.60 | 5.44E−02 | 2.00E−01 | | | |

TABLE 5

| | | |
|---|---|---|
| Genomic Location | | Reference Allele |
| (NCBI Build 35) | Reference | Frequency Difference |

| Chr | Position | Allele | D − L | AA − EA |
|---|---|---|---|---|
| 15 | 46179457 | A | −0.43 | −0.74 |
| 15 | 46258816 | A | 0.28 | 0.37 |
| 15 | 46056053 | G | 0.29 | 0.48 |
| 15 | 46420445 | C | 0.28 | 0.58 |
| 15 | 46098702 | A | −0.23 | −0.44 |
| 15 | 46087470 | T | −0.25 | −0.29 |
| 15 | 46473467 | A | −0.21 | −0.16 |
| 15 | 46049012 | C | 0.19 | 0.26 |
| 15 | 46097633 | G | −0.15 | −0.09 |
| 15 | 46051787 | T | 0.19 | 0.25 |
| 15 | 46472393 | C | 0.19 | 0.23 |
| 15 | 46306954 | G | −0.13 | 0.00 |
| 15 | 46157395 | G | −0.12 | −0.22 |
| 15 | 46971973 | G | 0.18 | 0.29 |
| 5 | 33987450 | C | 0.12 | 0.91 |
| 15 | 46986684 | G | 0.20 | 0.20 |
| 15 | 45957669 | C | 0.19 | 0.49 |
| 15 | 46861195 | A | −0.18 | −0.40 |
| 15 | 46843962 | T | −0.18 | −0.40 |
| 15 | 46827089 | T | −0.18 | −0.33 |
| 11 | 88551344 | C | 0.11 | 0.21 |
| 15 | 46521916 | G | 0.18 | 0.45 |
| 15 | 46039330 | T | 0.13 | 0.15 |
| 15 | 47018806 | G | 0.15 | −0.02 |
| 15 | 46055778 | G | 0.15 | 0.31 |
| 15 | 46930524 | G | 0.18 | 0.26 |
| 15 | 47009173 | G | 0.16 | 0.09 |
| 15 | 46979618 | T | −0.16 | −0.05 |
| 15 | 46569855 | T | −0.17 | −0.47 |
| 15 | 46963495 | A | −0.17 | −0.05 |
| 15 | 46731907 | C | 0.17 | 0.49 |
| 15 | 45903689 | T | −0.17 | −0.57 |
| 15 | 45922856 | C | −0.17 | −0.30 |
| 15 | 46791064 | T | 0.17 | 0.28 |
| 2 | 1.26E+08 | T | 0.16 | 0.22 |
| 15 | 45965538 | T | −0.14 | −0.11 |
| 2 | 1.26E+08 | A | 0.16 | 0.22 |
| 15 | 46892226 | A | 0.15 | 0.00 |
| 15 | 46089356 | G | 0.14 | 0.34 |
| 15 | 45970045 | G | −0.14 | −0.22 |
| 15 | 46800217 | G | 0.11 | 0.00 |
| 2 | 1.26E+08 | A | 0.16 | 0.22 |
| 18 | 36327583 | G | 0.12 | 0.16 |
| 15 | 46918903 | A | 0.15 | 0.28 |
| 7 | 19893304 | G | −0.13 | 0.09 |
| 14 | 23885591 | A | 0.14 | 0.53 |
| 15 | 25671898 | T | −0.14 | 0.06 |
| X | 1.09E+08 | A | 0.15 | 0.10 |
| 11 | 88159190 | C | 0.14 | 0.43 |
| 15 | 46034246 | A | −0.14 | −0.14 |
| 15 | 46834784 | A | −0.11 | 0.02 |
| 5 | 1.73E+08 | G | −0.14 | −0.23 |
| 14 | 82033421 | T | −0.14 | 0.07 |
| 15 | 45944278 | G | −0.12 | −0.15 |
| 8 | 17415668 | T | −0.13 | −0.33 |
| 15 | 46837115 | C | 0.11 | 0.02 |
| 14 | 82039583 | G | −0.14 | 0.06 |
| 11 | 88208027 | A | 0.14 | 0.49 |
| 16 | 4154994 | A | −0.13 | 0.02 |
| 15 | 44942842 | C | 0.14 | 0.12 |
| 15 | 46974966 | G | −0.12 | 0.16 |
| 3 | 1.25E+08 | C | 0.13 | 0.66 |
| X | 1.19E+08 | C | −0.15 | 0.00 |
| 11 | 88193818 | T | 0.14 | 0.49 |
| 15 | 44939404 | T | 0.14 | 0.04 |
| 15 | 58988635 | T | 0.14 | 0.50 |
| X | 33404293 | T | −0.13 | −0.27 |
| 2 | 76405736 | T | −0.14 | −0.01 |
| 6 | 48642656 | G | −0.11 | −0.21 |
| 2 | 2.09E+08 | A | 0.12 | 0.06 |
| 3 | 1.51E+08 | G | 0.12 | 0.46 |
| X | 1.09E+08 | A | 0.14 | −0.13 |
| 15 | 48059752 | C | −0.13 | −0.01 |
| 7 | 19751849 | C | −0.14 | −0.19 |
| 4 | 1.5E+08 | G | −0.12 | 0.12 |
| 15 | 70932755 | C | −0.12 | −0.12 |
| 18 | 59164885 | A | −0.13 | −0.41 |
| 7 | 19336733 | A | 0.13 | 0.33 |
| X | 79094700 | A | −0.12 | −0.60 |
| 3 | 1.51E+08 | T | 0.13 | 0.49 |
| 2 | 29343135 | A | −0.13 | −0.25 |
| X | 79145439 | G | −0.12 | −0.57 |
| 8 | 60745793 | G | −0.13 | −0.56 |
| 15 | 45925000 | A | −0.11 | −0.33 |
| 7 | 19389806 | A | −0.13 | −0.12 |
| 15 | 46939299 | C | −0.11 | −0.07 |
| 19 | 37719854 | A | 0.12 | 0.45 |
| 4 | 1.5E+08 | G | −0.11 | 0.13 |
| 11 | 88154001 | T | 0.12 | −0.07 |
| 6 | 90356598 | G | 0.12 | 0.38 |
| 12 | 22099211 | A | −0.13 | −0.25 |
| 2 | 1.04E+08 | C | −0.11 | −0.30 |
| 3 | 6463648 | T | −0.12 | −0.29 |
| 16 | 12368517 | T | 0.12 | 0.56 |
| 11 | 88193955 | G | 0.12 | −0.11 |
| 3 | 6463056 | T | −0.12 | −0.16 |
| 11 | 88160044 | C | 0.12 | 0.41 |
| 11 | 88191927 | C | 0.12 | −0.11 |
| 2 | 1.34E+08 | G | −0.11 | −0.36 |
| X | 1.02E+08 | C | 0.13 | 0.54 |
| 14 | 82064963 | T | −0.12 | −0.18 |
| 16 | 4380427 | C | 0.12 | −0.01 |
| 3 | 6463102 | T | −0.11 | −0.15 |
| 11 | 88207543 | T | 0.12 | −0.11 |
| 11 | 88207722 | A | 0.12 | −0.02 |
| 13 | 45063288 | A | 0.11 | 0.35 |
| 4 | 55196448 | G | −0.11 | −0.33 |
| 3 | 6491544 | T | 0.11 | 0.23 |
| 4 | 89895547 | C | 0.12 | 0.49 |
| 15 | 50659822 | G | −0.12 | −0.14 |
| 12 | 75951982 | T | −0.10 | 0.02 |
| 9 | 30862374 | T | −0.11 | 0.07 |
| 15 | 46950245 | T | 0.11 | 0.25 |
| 9 | 1344628 | A | 0.11 | 0.21 |
| X | 79148533 | T | −0.11 | 0.00 |
| X | 95453891 | A | −0.09 | −0.20 |
| X | 95532010 | A | −0.08 | −0.20 |
| X | 26730535 | A | −0.08 | 0.34 |
| 3 | 86466914 | T | 0.05 | 0.06 |
| 3 | 86398988 | A | 0.05 | −0.12 |

1. A method of screening a compound for activity in modulating tissue color, comprising
determining whether a compound binds to, modulates expression of, or modulates the activity of a polypeptide encoded by a gene shown in Table 2, column 3, 5 or 7.

2. The method of claim 1, wherein the gene is a gene shown in Table 2, column 3.

3. The method of claim 1, wherein the gene is selected from the group consisting of SLC24A5, LOC400369, MYEF2, DUT, and SLC12A1, RALP, and GRM5, preferably wherein the gene is SLC24A5.

4. The method of claim 1, wherein the gene is other than TYR, MATP, TYRP1, ADTB3A, DTNBP1, HPS1, HPS3, OA1, OCA2, MC1R, CDKN2A, MYO7A, EDN3, EDNRB, MITF, PAX3, SOX10, and KIT.

5. The method of claim 1, further comprising administering the compound to an animal and determining whether the compound modulates tissue color of the animal.

6. The method of claim 5, wherein the second determining step determines whether the compound modulates skin color of the animal.

7. The method of claim 5, wherein the second determining step determines whether the compound modulates eye color of the animal.

8. The method of claim 5, wherein the second determining step determines whether the compound modulates hair color of the animal.

9. The method of claim 1, wherein the determining comprises contacting the compound with the polypeptide and detecting specific binding between the compound and the polypeptide.

10. The method of claim 1, wherein the determining comprises contacting the compound with the polypeptide and detecting a modulation of activity of the polypeptide.

11. The method of claim 1, wherein the determining comprises contacting the gene or other nucleic acid encoding the polypeptide with the compound and detecting a modulation of expression of the polypeptide.

12-36. (canceled)

37. A method of polymorphic profiling an individual comprising
determining a polymorphic profile in at least two but no more than 1000 different haplotype blocks, and at least two of the haplotype blocks each comprising at least one gene shown in Table 2, columns 3, 5 or 7.

38. The method of claim 37, wherein the at least two haplotype blocks each comprise at least one gene shown in Table 2, column 3.

39. The method of claim 37, wherein the at least two haplotype blocks each comprise at least one gene selected from the group consisting of SLC24A5, LOC400369, MYEF2, DUT, and SLC12A1, RALP, and GRM5.

40. The method of claim 37, wherein the polymorphic profile is determined in at least 2 and no more than 50 different haplotype blocks.

41. The method of claim 37, wherein the polymorphic profile is determined in at least two SNPs at positions selected from the group consisting of 46213776, and 48013605.

42-79. (canceled)


SEQUENCE LISTING (190 sequences)

SEQ ID NO: 1 - protein, 25 aa, Artificial
  Zinc finger protein motif
  MISC_FEATURE (2)..(5): Residues 2 and 3 may be absent
  MISC_FEATURE (20)..(24): Residues 22 and 23 may be absent
  misc_feature (1)..(25): Xaa can be any naturally occurring amino acid
CXXXXCXXXXXXXXXXXXHXXXXXH

SEQ ID NO: 2 - DNA, 22 bp, Artificial
  Quantitative PCR primer for MATP
catgcctcctcactaccgctac

SEQ ID NO: 3 - DNA, 25 bp, Artificial
  Quantitative PCR primer for MATP
atctgtgaagaacagcatgttggac

SEQ ID NO: 4 - DNA, 22 bp, Artificial
  Quantitative PCR primer for MATP
tcatgctgaacagactcgcagg

SEQ ID NO: 5 - DNA, 23 bp, Artificial
  Quantitative PCR primer for MATP
tccatccaatgaggtggctgatg

SEQ ID NO: 6 - DNA, 24 bp, Artificial
  Quantitative PCR primer for SLC24A5
ccttggattgtctcaggatgttgc

SEQ ID NO: 7 - DNA, 25 bp, Artificial
  Quantitative PCR primer for SLC24A5
ggatggtgctaatgccaatatctcc

SEQ ID NO: 8 - DNA, 24 bp, Artificial
  Quantitative PCR primer for SLC12A1
gacctgctctcctggacataactc

SEQ ID NO: 9 - DNA, 25 bp, Artificial
  Quantitative PCR primer for SLC12A1
ccatgccactgttcatctccttaac

SEQ ID NO: 10 - DNA, 25 bp, Artificial
  Quantitative PCR primer for SLC12A1
ggaagatgatcaagctggtgttgtg

SEQ ID NO: 11 - DNA, 24 bp, Artificial
  Quantitative PCR primer for SLC12A1
aatccaggagaggcgaatgaagag

SEQ ID NO: 12 - DNA, 22 bp, Artificial
  Quantitative PCR primer for MYEF2
acagcctattagtgccagccag

SEQ ID NO: 13 - DNA, 24 bp, Artificial
  Quantitative PCR primer for MYEF2
gctattcattgcttccagaccacc

SEQ ID NO: 14 - DNA, 24 bp, Artificial
  Quantitative PCR primer for ATP8B4
cagtctgaagtgctcatcaacagc

SEQ ID NO: 15 - DNA, 24 bp, Artificial
  Quantitative PCR primer for ATP8B4
agagaccatgtggctcactacttg

SEQ ID NO: 16 - DNA, 19 bp, Artificial
  Quantitative PCR primer for DUT
ccacggtcaggcttggctg

SEQ ID NO: 17 - DNA, 24 bp, Artificial
  Quantitative PCR primer for DUT
aatgagctgtgcaattcgatcacc

SEQ ID NO: 18 - DNA, 22 bp, Artificial
  Quantitative PCR primer for SHC4
tgaaggcaaggtgaggaccaag

SEQ ID NO: 19 - DNA, 24 bp, Artificial
  Quantitative PCR primer for SHC4
taaggcttacttcgcttccagagg

SEQ ID NO: 20 - DNA, 21 bp, Artificial
  Quantitative PCR primer for GRM5
ggcatgacggtgagaggtctg

SEQ ID NO: 21 - DNA, 24 bp, Artificial
  Quantitative PCR primer for GRM5
tctgtcacatcatacctgtcagcc

SEQ ID NO: 22 - DNA, 23 bp, Artificial
  Quantitative PCR primer for DRG2
cctggactatctgctggagatgc

SEQ ID NO: 23 - DNA, 22 bp, Artificial
  Quantitative PCR primer for DRG2
tgatggcgtctgtgaagtctgg

SEQ ID NO: 24 - DNA, 24 bp, Artificial
  Quantitative PCR primer for MYO 15a
gagcagatagactggcaggagatc

SEQ ID NO: 25 - DNA, 23 bp, Artificial
  Quantitative PCR primer for MYO 15a
tggcacttctgtaggaaggtgtg

SEQ ID NO: 26 - DNA, 20 bp, Artificial
  Quantitative PCR primer for CRI-1
agccgctcttgaagaagccg

SEQ ID NO: 27 - DNA, 25 bp, Artificial
  Quantitative PCR primer for CRI-1
gtcagacgattgacaaccatcagtg

SEQ ID NO: 28 - DNA, 25 bp, Artificial
  Quantitative PCR primer for MYLK
atcacctgtacctggatgaagttcc

SEQ ID NO: 29 - DNA, 22 bp, Artificial
  Quantitative PCR primer for MYLK
cttgctgccattctcgctgttc

SEQ ID NO: 30 - DNA, 21 bp, Artificial
  Quantitative PCR primer for ALK
ggatggtgttgcctctcctcg

SEQ ID NO: 31 - DNA, 24 bp, Artificial
  Quantitative PCR primer for ALK
atcttgtcctctccgctaatggtg

SEQ ID NO: 32 - DNA, 25 bp, Artificial
  Quantitative PCR primer for DDB1
gcttatccagatcacttcagcatcg

SEQ ID NO: 33 - DNA, 20 bp, Artificial
  Quantitative PCR primer for DDB1
ctgcctacagccaccaccac

SEQ ID NO: 34 - DNA, 25 bp, Artificial
  Quantitative PCR primer for FBN1
gctggtggtgagtgtattaacaacc

SEQ ID NO: 35 - DNA, 25 bp, Artificial
  Quantitative PCR primer for FBN1
ctcatcaatgtctcggcattctgtc

SEQ ID NO: 36 - DNA, 23 bp, Artificial
  Quantitative PCR primer for Sema6D
cgactggaaatgctttacggaag

SEQ ID NO: 37 - DNA, 21 bp, Artificial
  Quantitative PCR primer for Sema6D
cgtaacacatctcagcaccga

SEQ ID NO: 38 - DNA, 29 bp, Artificial
  29-mer comprising polymorphic site
acctcagaaaccacracataaaccaagga

SEQ ID NO: 39 - DNA, 29 bp, Artificial
  29-mer comprising polymorphic site
actcagttcaaatayaatctcttgcaaga

SEQ ID NO: 40 - DNA, 29 bp, Artificial
  29-mer comprising polymorphic site
aaagtaatactcaawtaacataatttcat

SEQ ID NO: 41 - DNA, 29 bp, Artificial
  29-mer comprising polymorphic site
ccattcctggggatragaagccagtaaca

SEQ ID NO: 42 - DNA, 29 bp, Artificial
  29-mer comprising polymorphic site
tttaaaacccaaatygtaattttctccta

SEQ ID NO: 43 - DNA, 29 bp, Artificial
  29-mer comprising polymorphic site
ttccccaattcactmcctgctcagactgt

SEQ ID NO: 44 - DNA, 29 bp, Artificial
  29-mer comprising polymorphic site
gcaaagtagaggagyagatggatcaggaa

SEQ ID NO: 45 - DNA, 29 bp, Artificial
  29-mer comprising polymorphic site
tgatttctccattcrttgcttggctctta

SEQ ID NO: 46 - DNA, 29 bp, Artificial
  29-mer comprising polymorphic site
accattccatgttayggtgtttctgccaa

SEQ ID NO: 47 - DNA, 29 bp, Artificial
  29-mer comprising polymorphic site
atttggttgcatccracaccaggcaaggg

SEQ ID NO: 48 - DNA, 29 bp, Artificial
  29-mer comprising polymorphic site
caaaaacccattcakattcaagggattat

SEQ ID NO: 49 - DNA, 29 bp, Artificial
  29-mer comprising polymorphic site
ctcttcgccctctcyggggatgttcgggt

SEQ ID NO: 50 - DNA, 29 bp, Artificial
  29-mer comprising polymorphic site
ttctttgtaccttgsatgagacccactgg

SEQ ID NO: 51 - DNA, 29 bp, Artificial
  29-mer comprising polymorphic site
attacggtcatgatkaactgaaaccctta

SEQ ID NO: 52 - DNA, 29 bp, Artificial
  29-mer comprising polymorphic site
ggataacacagatarttgggccctctggc

SEQ ID NO: 53 - DNA, 29 bp, Artificial
  29-mer comprising polymorphic site
acggagttgatgcasaagccccaacatcc

SEQ ID NO: 54 - DNA, 29 bp, Artificial
  29-mer comprising polymorphic site
tgataacggtcatgrtgatgtgtgatttc

SEQ ID NO: 55 - DNA, 29 bp, Artificial
  29-mer comprising polymorphic site
gttgattgtttatgktatttatgcatgtg

SEQ ID NO: 56 - DNA, 29 bp, Artificial
  29-mer comprising polymorphic site
gcctgaccttgaatsaagccatttattct

SEQ ID NO: 57 - DNA, 29 bp, Artificial
  29-mer comprising polymorphic site
ggtttgccaagaacrggttgtactttagc

SEQ ID NO: 58 - DNA, 29 bp, Artificial
  29-mer comprising polymorphic site
acttgttgtgcgtgkcttggatagcaaaa

SEQ ID NO: 59 - DNA, 29 bp, Artificial
  29-mer comprising polymorphic site
tgaatcctaaaggaygagagtaagactaa

SEQ ID NO: 60 - DNA, 29 bp, Artificial
  29-mer comprising polymorphic site
actgcttgggggatmtgaaatctggagag

SEQ ID NO: 61 - DNA, 29 bp, Artificial
  29-mer comprising polymorphic site
tcatagaagatgacrctcctgatttgtgg

SEQ ID NO: 62 - DNA, 29 bp, Artificial
  29-mer comprising polymorphic site
ctccctagagtagawtgtggtttgagaga

SEQ ID NO: 63 - DNA, 29 bp, Artificial
  29-mer comprising polymorphic site
gtcagaggaaggacrctggggcgagttat

SEQ ID NO: 64 - DNA, 29 bp, Artificial
  29-mer comprising polymorphic site
tgtgtgcccaagaakaaagggtaaacact

SEQ ID NO: 65 - DNA, 29 bp, Artificial
  29-mer comprising polymorphic site
ttctgggggtgttarttttgctgagtagg

SEQ ID NO: 66 - DNA, 29 bp, Artificial
  29-mer comprising polymorphic site
atatgcaacattctkggcctatctgagaa

SEQ ID NO: 67 - DNA, 29 bp, Artificial
  29-mer comprising polymorphic site
aacagcagatgtgartccaaactgctctg

SEQ ID NO: 68 - DNA, 29 bp, Artificial
  29-mer comprising polymorphic site
aattttgttttcaaygtagtcactctata

SEQ ID NO: 69 - DNA, 29 bp, Artificial
  29-mer comprising polymorphic site
gaagcataaattatstaagtcatcttaca

SEQ ID NO: 70 - DNA, 29 bp, Artificial
  29-mer comprising polymorphic site
gagatcccacagtgytctttcgggagatg

SEQ ID NO: 71 - DNA, 29 bp, Artificial
  29-mer comprising polymorphic site
ctatgttctttgcawcttagttcttcatt

SEQ ID NO: 72 - DNA, 29 bp, Artificial
  29-mer comprising polymorphic site
tcacccagggacccyatccacaaaatgca

SEQ ID NO: 73 - DNA, 29 bp, Artificial
  29-mer comprising polymorphic site
tgcccatgtgcacaycaaggtagacaaac

SEQ ID NO: 74 - DNA, 29 bp, Artificial
  29-mer comprising polymorphic site
aaaagtcattgttgytaaagcgggtcaac

SEQ ID NO: 75 - DNA, 29 bp, Artificial
  29-mer comprising polymorphic site
tcaatccctttagcygttttctagtattt

SEQ ID NO: 76 - DNA, 29 bp, Artificial
  29-mer comprising polymorphic site
gacccattgactaakaaacatttttgttg

SEQ ID NO: 77 - DNA, 29 bp, Artificial
  29-mer comprising polymorphic site
tctggggaagggaawtggcattggaacat

SEQ ID NO: 78 - DNA, 29 bp, Artificial
  29-mer comprising polymorphic site
gggaccatctacaarcattatttttttaa

SEQ ID NO: 79 - DNA, 29 bp, Artificial
  29-mer comprising polymorphic site
tttacagattggtawattctttcacaagc

SEQ ID NO: 80 - DNA, 29 bp, Artificial
  29-mer comprising polymorphic site
gatgcatactaagtraggggagagttcta

SEQ ID NO: 81 - DNA, 29 bp, Artificial
  29-mer comprising polymorphic site
ttgggatgggagaasagctgccaagtcag

SEQ ID NO: 82 - DNA, 29 bp, Artificial
  29-mer comprising polymorphic site
attgattattctctstgctgcacctatat

SEQ ID NO: 83 - DNA, 29 bp, Artificial
  29-mer comprising polymorphic site
tatttgtgtgggaaragctttcaaagcct

SEQ ID NO: 84 - DNA, 29 bp, Artificial
  29-mer comprising polymorphic site
atattcccttaatcrgaaaagagagtgac

SEQ ID NO: 85 - DNA, 29 bp, Artificial
  29-mer comprising polymorphic site
tttctccaacatctrctttaagtatgcac

SEQ ID NO: 86 - DNA, 29 bp, Artificial
  29-mer comprising polymorphic site
tcactacaaaaacartatgaatatgatac

SEQ ID NO: 87 - DNA, 29 bp, Artificial
  29-mer comprising polymorphic site
ttaacgttttttctrccacaattgctaca

SEQ ID NO: 88 - DNA, 29 bp, Artificial
  29-mer comprising polymorphic site
ttgctgtgtttccartatgaagaacatat

SEQ ID NO: 89 - DNA, 29 bp, Artificial
  29-mer comprising polymorphic site
aggggaatttaaaaygtcctaggccaatg

SEQ ID NO: 90 - DNA, 29 bp, Artificial
  29-mer comprising polymorphic site
gtctcagtttgaagragtgataaataaat

SEQ ID NO: 91 - DNA, 29 bp, Artificial
  29-mer comprising polymorphic site
aagaattcttaatgyattgctttgccatg

SEQ ID NO: 92 - DNA, 29 bp, Artificial
  29-mer comprising polymorphic site
ccatgctgagcagargaattacaagcaat

SEQ ID NO: 93 - DNA, 29 bp, Artificial
  29-mer comprising polymorphic site
aacatatatgtgtaragcaaaaatatttt

SEQ ID NO: 94 - DNA, 29 bp, Artificial
  29-mer comprising polymorphic site
atttgcttgtttctrtatcaatacctttg

SEQ ID NO: 95 - DNA, 29 bp, Artificial
  29-mer comprising polymorphic site
agaagtgattttccrgcgagaagcagcgg

SEQ ID NO: 96 - DNA, 29 bp, Artificial
  29-mer comprising polymorphic site
ataccaataatcatktatgatacactttc

SEQ ID NO: 97 - DNA, 29 bp, Artificial
  29-mer comprising polymorphic site
acaaaggtgcttacrttgtgaataatgac

SEQ ID NO: 98 - DNA, 29 bp, Artificial
  29-mer comprising polymorphic site
ttgaccaagcaaaaktgactttttgtccc

SEQ ID NO: 99 - DNA, 29 bp, Artificial
  29-mer comprising polymorphic site
gtcaaaagacagaaytgggcatctccaaa

SEQ ID NO: 100 - DNA, 29 bp, Artificial
  29-mer comprising polymorphic site
tgtgagagactgagrataagcagaaaagg

SEQ ID NO: 101 - DNA, 29 bp, Artificial
  29-mer comprising polymorphic site
tggaaatgtcttacrtgataaacctgata

SEQ ID NO: 102 - DNA, 29 bp, Artificial
  29-mer comprising polymorphic site
gttttgcgactccawactgatcaccgttg

SEQ ID NO: 103 - DNA, 29 bp, Artificial
  29-mer comprising polymorphic site
ctaatttctgtcacyggacttaaattcag

SEQ ID NO: 104 - DNA, 29 bp, Artificial
  29-mer comprising polymorphic site
aaataggcttgtacwatccatctattaat

SEQ ID NO: 105 - DNA, 29 bp, Artificial
  29-mer comprising polymorphic site
tcgttaatacccgcrtggctggtaaacta

SEQ ID NO: 106 - DNA, 29 bp, Artificial
  29-mer comprising polymorphic site
tacaaacccaggtcytgctcataggcatt

SEQ ID NO: 107 - DNA, 29 bp, Artificial
  29-mer comprising polymorphic site
cgttgtgttcactaycatagtgtcagtgc

SEQ ID NO: 108 - DNA, 29 bp, Artificial
  29-mer comprising polymorphic site
ataagacatcatttyagaaatatatacaa

SEQ ID NO: 109 - DNA, 29 bp, Artificial
  29-mer comprising polymorphic site
ttgtataacagagcyataagaaataagac

SEQ ID NO: 110 - DNA, 29 bp, Artificial
  29-mer comprising polymorphic site
aattcttgctgtggwcccagcggtgagca

SEQ ID NO: 111 - DNA, 29 bp, Artificial
  29-mer comprising polymorphic site
gaaacatctgaaaaycaaattatcaaagt

SEQ ID NO: 112 - DNA, 29 bp, Artificial
  29-mer comprising polymorphic site
ttaatttatactacktctagaaacaaaca

SEQ ID NO: 113 - DNA, 29 bp, Artificial
  29-mer comprising polymorphic site
aaagtgttcttcaayattcatactacttt

SEQ ID NO: 114 - DNA, 29 bp, Artificial
  29-mer comprising polymorphic site
tcccagtcaaggcasgtaggatccctatt

SEQ ID NO: 115 - DNA, 29 bp, Artificial
  29-mer comprising polymorphic site
tcatcaatataaatrttctccaagtttat

SEQ ID NO: 116 - DNA, 29 bp, Artificial
  29-mer comprising polymorphic site
ctctctctctctttrggattctaaggata

SEQ ID NO: 117 - DNA, 29 bp, Artificial
  29-mer comprising polymorphic site
aataatagcctattktatacaacccaact

SEQ ID NO: 118 - DNA, 29 bp, Artificial
  29-mer comprising polymorphic site
atgcatcctcttggwtaaggattcctgta

SEQ ID NO: 119 - DNA, 29 bp, Artificial
  29-mer comprising polymorphic site
taattaccttctttycttattcagagtcc

SEQ ID NO: 120 - DNA, 29 bp, Artificial
  29-mer comprising polymorphic site
attttttcaagggcmaagattattacata

SEQ ID NO: 121 - DNA, 29 bp, Artificial
  29-mer comprising polymorphic site
ctgtcttcactgatrccatgttgtttgag

SEQ ID NO: 122 - DNA, 29 bp, Artificial
  29-mer comprising polymorphic site
cttccaatcaaacamcctccaatcattct

SEQ ID NO: 123 - DNA, 29 bp, Artificial
  29-mer comprising polymorphic site
tcgcttcctagattrgtattctcgctatg

SEQ ID NO: 124 - DNA, 29 bp, Artificial
  29-mer comprising polymorphic site
ttaccagcctatccrttttctgacaagtt

SEQ ID NO: 125 - DNA, 29 bp, Artificial
  29-mer comprising polymorphic site
agtcccctgcttctragtaagtgactcat

SEQ ID NO: 126 - DNA, 29 bp, Artificial
  29-mer comprising polymorphic site
tccatttaagtgaaygggtaaggcctccc

SEQ ID NO: 127 - DNA, 29 bp, Artificial
  29-mer comprising polymorphic site
tgccccgtggtaacrtgatggcctcagca

SEQ ID NO: 128 - DNA, 29 bp, Artificial
  29-mer comprising polymorphic site
cttgatagtaggctrtacaactgttcaat

SEQ ID NO: 129 - DNA, 29 bp, Artificial
  29-mer comprising polymorphic site
atactttttaaaaakgatgacatgataaa

SEQ ID NO: 130 - DNA, 29 bp, Artificial
  29-mer comprising polymorphic site
cagttttctagtctygatatttttcttta

SEQ ID NO: 131 - DNA, 29 bp, Artificial
  29-mer comprising polymorphic site
gtgagaaaaaagaarttgactgagcaaat

SEQ ID NO: 132 - DNA, 29 bp, Artificial
  29-mer comprising polymorphic site
cactacaaacatatwcaccaattataaaa

SEQ ID NO: 133 - DNA, 29 bp, Artificial
  29-mer comprising polymorphic site
cttgctagtcaggcstcatatccggagac

SEQ ID NO: 134 - DNA, 29 bp, Artificial
  29-mer comprising polymorphic site
ttcgccaaaagtaaratactattaccaga

SEQ ID NO: 135 - DNA, 29 bp, Artificial
  29-mer comprising polymorphic site
actccttccactacrtgataccttcagct

SEQ ID NO: 136 - DNA, 29 bp, Artificial
  29-mer comprising polymorphic site
tgtgcagaggcttaytttgaagagcatgt

SEQ ID NO: 137 - DNA, 29 bp, Artificial
  29-mer comprising polymorphic site
tactctgaagatctsggaagctgtaggtt

SEQ ID NO: 138 - DNA, 29 bp, Artificial
  29-mer comprising polymorphic site
tgttacagatccaarggagtataaaatgt

SEQ ID NO: 139 - DNA, 29 bp, Artificial
  29-mer comprising polymorphic site
tgatagaaaggcaasgatgttgtgaggaa

SEQ ID NO: 140 - DNA, 29 bp, Artificial
  29-mer comprising polymorphic site
atgacatcgtactaygttgaaaagtggcc

SEQ ID NO: 141 - DNA, 29 bp, Artificial
  29-mer comprising polymorphic site
cactttgtgctgtgktgtttgccactctg

SEQ ID NO: 142 - DNA, 29 bp, Artificial
  29-mer comprising polymorphic site
taaaatggccatagrtaagaagataatta

SEQ ID NO: 143 - DNA, 29 bp, Artificial
  29-mer comprising polymorphic site
ccagcatgtaaaaayagagacatttccaa

SEQ ID NO: 144 - DNA, 29 bp, Artificial
  29-mer comprising polymorphic site
gataagctgagatcygacatccaagcatc

SEQ ID NO: 145 - DNA, 29 bp, Artificial
  29-mer comprising polymorphic site
atcactaacaagaaygcttccaaagagag

SEQ ID NO: 146 - DNA, 29 bp, Artificial
  29-mer comprising polymorphic site
cataaaaaaccaaastaggaaaagggaaa

SEQ ID NO: 147 - DNA, 29 bp, Artificial
  29-mer comprising polymorphic site
atatgaagcaggtaygtcaaatcaatgtc

SEQ ID NO: 148 - DNA, 29 bp, Artificial
  29-mer comprising polymorphic site
ggcttccttagacayattgaaatagtcat

SEQ ID NO: 149 - DNA, 29 bp, Artificial
  29-mer comprising polymorphic site
tgcatgtctcacagstgatagcagggtac

SEQ ID NO: 150 - DNA, 29 bp, Artificial
  29-mer comprising polymorphic site
ttgtattttgcagayttgtagtgaaattg

SEQ ID NO: 151 - DNA, 29 bp, Artificial
  29-mer comprising polymorphic site
agtgctcatttcttyagacgtgatttgca

SEQ ID NO: 152 - DNA, 29 bp, Artificial
  29-mer comprising polymorphic site
ttcgattttagggtrtgagaatcctgcct

SEQ ID NO: 153 - DNA, 29 bp, Artificial
  29-mer comprising polymorphic site
gataaattatgccamcaattctgataata

SEQ ID NO: 154 - DNA, 29 bp, Artificial
  29-mer comprising polymorphic site
agtttgatcagagaragctgcccagaggg

SEQ ID NO: 155 - DNA, 29 bp, Artificial
  29-mer comprising polymorphic site
atttctgaatctcaytggcatttttctaa

SEQ ID NO: 156 - DNA, 29 bp, Artificial
  29-mer comprising polymorphic site
aattgagaaccttcyctgaggacaagtca

SEQ ID NO: 157 - DNA, 29 bp, Artificial
  29-mer comprising polymorphic site
aaagtgctcagaaasgttggaagactgtt

SEQ ID NO: 158 - DNA, 29 bp, Artificial
  29-mer comprising polymorphic site
caaatcaaaagataytcagtttgccactg

SEQ ID NO: 159 - DNA, 29 bp, Artificial
  29-mer comprising polymorphic site
ggtcatttgtccttktttgctccacaacc

SEQ ID NO: 160 - DNA, 29 bp, Artificial
  29-mer comprising polymorphic site
tgaagacaagtaatyattgaagtgttttt

SEQ ID NO: 161 - DNA, 29 bp, Artificial
  29-mer comprising polymorphic site
aaggaacccactacrtagcaacccaattt

SEQ ID NO: 162 - DNA, 29 bp, Artificial
  29-mer comprising polymorphic site
tttctacaaggacarttctttgcctttag

SEQ ID NO: 163 - DNA, 29 bp, Artificial
  29-mer comprising polymorphic site
ttttatgactgtgcrtccatcaaatttac

SEQ ID NO: 164 - DNA, 29 bp, Artificial
  29-mer comprising polymorphic site
tataatttcctcaaygtcaaaactaacag

SEQ ID NO: 165 - DNA, 29 bp, Artificial
  29-mer comprising polymorphic site
gaacaccgtcttggrtgtcaaaaagactt

SEQ ID NO: 166 - DNA, 29 bp, Artificial
  29-mer comprising polymorphic site
taatactagacaacrtggtaatgatagga

SEQ ID NO: 167 - DNA, 29 bp, Artificial
  29-mer comprising polymorphic site
tatttgacatctctrtaattttcctatct

SEQ ID NO: 168 - DNA, 29 bp, Artificial
  29-mer comprising polymorphic site
tattttttacattakaaatctcctgaatt

SEQ ID NO: 169 - DNA, 29 bp, Artificial
  29-mer comprising polymorphic site
agccactaataattrctttttcagtgaat

SEQ ID NO: 170 - DNA, 29 bp, Artificial
  29-mer comprising polymorphic site
aggatgttgcaggcrcaactttcatggca

SEQ ID NO: 171 - DNA, 29 bp, Artificial
  29-mer comprising polymorphic site
gatttagaacatatytgttattagctatg

SEQ ID NO: 172 - DNA, 29 bp, Artificial
  29-mer comprising polymorphic site
atgacagtgagtttwgccagctggaacca

SEQ ID NO: 173 - DNA, 29 bp, Artificial
  29-mer comprising polymorphic site
ctgattaacaaaccrttagtaattccctt

SEQ ID NO: 174 - DNA, 29 bp, Artificial
  29-mer comprising polymorphic site
aaagttctgctttaytactactgtctctt

SEQ ID NO: 175 - DNA, 29 bp, Artificial
  29-mer comprising polymorphic site
taccctggctctagyccactagctccttc

SEQ ID NO: 176 - DNA, 29 bp, Artificial
  29-mer comprising polymorphic site
ggggttggtattgayaaggccacctgagg

SEQ ID NO: 177 - DNA, 29 bp, Artificial
  29-mer comprising polymorphic site
taggagtatagagarcaactttgagcaat

SEQ ID NO: 178 - DNA, 29 bp, Artificial
  29-mer comprising polymorphic site
acagagtttctgctytttcacttgcttag

SEQ ID NO: 179 - DNA, 29 bp, Artificial
  29-mer comprising polymorphic site
agaagtatagcaggstttatgtagaccag

SEQ ID NO: 180 - DNA, 29 bp, Artificial
  29-mer comprising polymorphic site
ttattagcagttagytgaaacaacagatt

SEQ ID NO: 181 - DNA, 29 bp, Artificial
  29-mer comprising polymorphic site
tcttactacaacagmaacattttaaaaag

SEQ ID NO: 182 - DNA, 29 bp, Artificial
  29-mer comprising polymorphic site
caggaaattgctcartatgggagacttag

SEQ ID NO: 183 - DNA, 29 bp, Artificial
  29-mer comprising polymorphic site
ctggtctgaatctgsaatgctgtatggct

SEQ ID NO: 184 - DNA, 29 bp, Artificial
  29-mer comprising polymorphic site
tgtgcccaaactccraagttttttccaat

SEQ ID NO: 185 - DNA, 29 bp, Artificial
  29-mer comprising polymorphic site
accctcccaaggtgkgctcacattaaatg

SEQ ID NO: 186 - DNA, 29 bp, Artificial
  29-mer comprising polymorphic site
tgacagtggattacraggccacaccatga

SEQ ID NO: 187 - DNA, 29 bp, Artificial
  29-mer comprising polymorphic site
agatgttctaatacytgtcttcctccaga

SEQ ID NO: 188 - DNA, 29 bp, Artificial
  29-mer comprising polymorphic site
gtcaggacacagctyggggtcacggcgca

SEQ ID NO: 189 - DNA, 29 bp, Artificial
  29-mer comprising polymorphic site
ggattccatcagatkgtggtcaaagaact

SEQ ID NO: 190 - DNA, 29 bp, Artificial
  29-mer comprising polymorphic site
ttattgttactcacytccattgctactag